SEQUENCE LISTING (54 sequences)

SEQ ID NO: 1 - DNA, 2196 bp, Homo sapiens
atgtctggtagccgccaggccgggtcgggctccgctgggacaagccccgggtcctcggcggcctcctcggtgacttccgcctcctcgtctttatcctcttccccgtcgccgccttccgtggcggtttcggcggcagcgctggtgtccggcggggtggcccaggccgccggctcgggcggcctcgggggcccggtgcggcctgtgttggtggcgcccgccgtatcgggtagcggcggcggggcggtgtccacgggcctgtcccggcacagctgcgcggccaggcccagcgccggcgtaggaggcagcagctccagcctaggcagcggcagcaggaagcgacctctcctcgcccccctctgcaacgggctcatcaactcctacgaggacaaaagcaacgacttcgtatgccccatctgctttgatatgattgaagaagcatacatgacaaaatgtggccacagcttttgctacaagtgtattcatcagagtttggaggacaataatagatgtcccaagtgtaactatgttgtggacaatattgaccatctgtatcctaatttcttggtgaatgaactcattcttaaacagaagcaaagatttgaggaaaagaggttcaaattggaccactcagtgagtagcaccaatggccacaggtggcagatatttcaagattggttgggaactgaccaagataaccttgatttggccaatgtcaatcttatgttggagttactagtgcagaagaagaaacaactggaagcagaatcacatgcagcccaactacagattcttatggaattcctcaaggttgcaagaagaaataagagagagcaactggaacagatccagaaggagctaagtgttttggaagaggatattaagagagtggaagaaatgagtggcttatactctcctgtcagtgaggatagcacagtgcctcaatttgaagctccttctccatcacacagtagtattattgattccacagaatacagccaacctccaggtttcagtggcagttctcagacaaagaaacagccttggtataatagcacgttagcatcaagacgaaaacgacttactgctcattttgaagacttggagcagtgttacttttctacaaggatgtctcgtatctcagatgacagtcgaactgcaagccagttggatgaatttcaggaatgcttgtccaagtttactcgatataattcagtacgacctttagccacattgtcatatgctagtgatctctataatggttccagtatagtctctagtattgaatttgaccgggattgtgactattttgcgattgctggagttacaaagaagattaaagtctatgaatatgacactgtcatccaggatgcagtggatattcattaccctgagaatgaaatgacctgcaattcgaaaatcagctgtatcagttggagtagttaccataagaacctgttagctagcagtgattatgaaggcactgttattttatgggatggattcacaggacagaggtcaaaggtctatcaggagcatgagaagaggtgttggagtgttgactttaatttgatggatcctaaactcttggcttcaggttctgatgatgcaaaagtgaagctgtggtctaccaatctagacaactcagtggcaagcattgaggcaaaggctaatgtgtgctgtgttaaattcagcccctcttccagataccatttggctttcggctgtgcagatcactgtgtccactactatgatcttcgtaacactaaacagccaatcatggtattcaaaggacaccgtaaagcagtctcttatgcaaagtttgtgagtggtgaggaaattgtctctgcctcaacagacagtcagctaaaactgtggaatgtagggaaaccatactgcctacgttccttcaagggtcatatcaatgaaaaaaactttgtaggcctggcttccaatggagattatatagcttgtggaagtgaaaataactctctctacctgtactataaaggactttctaagactttgctaacttttaagtttgatacagtcaaaagtgttctcgacaaagaccgaaaagaagatgatacaaatgaatttgttagtgctgtgtgctggagggcactaccagatggggagtccaatgtgctgattgctgctaacagtcagggtacaattaaggtgctagaattggtatga

SEQ ID NO: 2 - protein, 731 aa, Homo sapiens
MSGSRQAGSGSAGTSPGSSAASSVTSASSSLSSSPSPPSVAVSAAALVSGGVAQAAGSGGLGGPVRPVLVAPAVSGSGGGAVSTGLSRHSCAARPSAGVGGSSSSLGSGSRKRPLLAPLCNGLINSYEDKSNDFVCPICFDMIEEAYMTKCGHSFCYKCIHQSLEDNNRCPKCNYVVDNIDHLYPNFLVNELILKQKQRFEEKRFKLDHSVSSTNGHRWQIFQDWLGTDQDNLDLANVNLMLELLVQKKKQLEAESHAAQLQILMEFLKVARRNKREQLEQIQKELSVLEEDIKRVEEMSGLYSPVSEDSTVPQFEAPSPSHSSIIDSTEYSQPPGFSGSSQTKKQPWYNSTLASRRKRLTAHFEDLEQCYFSTRMSRISDDSRTASQLDEFQECLSKFTRYNSVRPLATLSYASDLYNGSSIVSSIEFDRDCDYFAIAGVTKKIKVYEYDTVIQDAVDIHYPENEMTCNSKISCISWSSYHKNLLASSDYEGTVILWDGFTGQRSKVYQEHEKRCWSVDFNLMDPKLLASGSDDAKVKLWSTNLDNSVASIEAKANVCCVKFSPSSRYHLAFGCADHCVHYYDLRNTKQPIMVFKGHRKAVSYAKFVSGEEIVSASTDSQLKLWNVGKPYCLRSFKGHINEKNFVGLASNGDYIACGSENNSLYLYYKGLSKTLLTFKFDTVKSVLDKDRKEDDTNEFVSAVCWRALPDGESNVLIAANSQGTIKVLELV

SEQ ID NO: 3 - protein, 12 aa, Homo sapiens
DSRTASQLDEFC

SEQ ID NO: 4 - protein, 6 aa, Homo sapiens
SRTASQ

SEQ ID NO: 5 - protein, 5 aa, Homo sapiens
RTASQ

SEQ ID NO: 6 - protein, 4 aa, Homo sapiens
TASQ

SEQ ID NO: 7 - protein, 3 aa, Homo sapiens
ASQ

SEQ ID NO: 8 - protein, 5 aa, Homo sapiens
SQLDE

SEQ ID NO: 9 - protein, 4 aa, Homo sapiens
SQLD

SEQ ID NO: 10 - protein, 3 aa, Homo sapiens
SQL

SEQ ID NO: 11 - protein, 4 aa, Homo sapiens
ASQL

SEQ ID NO: 12 - protein, 6 aa, Homo sapiens
TASQLD

SEQ ID NO: 13 - protein, 8 aa, Homo sapiens
RTASQLDE

SEQ ID NO: 14 - protein, 10 aa, Homo sapiens
SRTASQLDEF

SEQ ID NO: 15 - protein, 16 aa, Homo sapiens
DSRTASTYDEQLDEFC

SEQ ID NO: 16 - protein, 10 aa, Homo Sapiens
SRTASTYDEQ

SEQ ID NO: 17 - protein, 9 aa, Homo sapiens
RTASTYDEQ

SEQ ID NO: 18 - protein, 8 aa, Homo sapiens
TASTYDEQ

SEQ ID NO: 19 - protein, 7 aa, Homo sapiens
ASTYDEQ

SEQ ID NO: 20 - protein, 9 aa, Homo sapiens
STYDEQLDE

SEQ ID NO: 21 - protein, 8 aa, Homo sapiens
STYDEQLD

SEQ ID NO: 22 - protein, 7 aa, Homo sapiens
STYDEQL

SEQ ID NO: 23 - protein, 8 aa, Homo sapiens
ASTYDEQL

SEQ ID NO: 24 - protein, 10 aa, Homo sapiens
TASTYDEQLD

SEQ ID NO: 25 - protein, 12 aa, Homo sapiens
RTASTYDEQLDE

SEQ ID NO: 26 - protein, 14 aa, Homo sapiens
SRTASTYDEQLDEF

SEQ ID NO: 27 - protein, 6 aa, Homo sapiens
SRTASQ

SEQ ID NO: 28 - protein, 5 aa, Homo sapiens
RTASQ

SEQ ID NO: 29 - protein, 4 aa, Homo sapiens
TASQ

SEQ ID NO: 30 - protein, 3 aa, Homo sapiens
ASQ

SEQ ID NO: 31 - protein, 5 aa, Homo sapiens
SQLDE

SEQ ID NO: 32 - protein, 4 aa, Homo sapiens
SQLD

SEQ ID NO: 33 - protein, 3 aa, Homo sapiens
SQL

SEQ ID NO: 34 - protein, 4 aa, Homo sapiens
ASQL

SEQ ID NO: 35 - protein, 6 aa, Homo sapiens
TASQLD

SEQ ID NO: 36 - protein, 8 aa, Homo sapiens
RTASQLDE

SEQ ID NO: 37 - protein, 10 aa, Homo sapiens
SRTASQLDEF

SEQ ID NO: 38 - protein, 6 aa, Homo sapiens
SRTASQ

SEQ ID NO: 39 - protein, 5 aa, Homo sapiens
RTASQ

SEQ ID NO: 40 - protein, 4 aa, Homo sapiens
TASQ

SEQ ID NO: 41 - protein, 3 aa, Homo sapiens
ASQ

SEQ ID NO: 42 - protein, 5 aa, Homo sapiens
SQLDE

SEQ ID NO: 43 - protein, 4 aa, Homo sapiens
SQLD

SEQ ID NO: 44 - protein, 3 aa, Homo sapiens
SQL

SEQ ID NO: 45 - protein, 4 aa, Homo sapiens
ASQL

SEQ ID NO: 46 - protein, 6 aa, Homo sapiens
TASQLD

SEQ ID NO: 47 - protein, 8 aa, Homo sapiens
RTASQLDE

SEQ ID NO: 48 - protein, 10 aa, Homo sapiens
SRTASQLDEF

SEQ ID NO: 49 - protein, 16 aa, Homo sapiens
SDDSRTASQLDEFQEC

SEQ ID NO: 50 - protein, 16 aa, mus musculus
SDDSRTASQLDEFQEC

SEQ ID NO: 51 - protein, 16 aa, Rattus norvegicus
SDDSRTASQLDEFQEC

SEQ ID NO: 52 - protein, 16 aa, Canis
SDDSRTASQLDEFQEC

SEQ ID NO: 53 - protein, 16 aa, Gallus domesticus
SDDSRTTSQLDEFQEC

SEQ ID NO: 54 - protein, 16 aa, Rana
SDDSRTASQLDEFQEC

(19) United States
(12) Patent Application Publication
(10) Pub. No.: US 2008/0027004 A1
(43) Pub. Date: Jan. 31, 2008

(54) Phosphorylated COP1 molecules and uses thereof

(75) Inventor: David Dornan, Burlingame, CA (US)

Correspondence Address: HELLER EHRMAN LLP, 275 MIDDLEFIELD ROAD, MENLO PARK, CA, 94025-3506, US

(73) Assignee: Genentech, Inc., South San Francisco, CA (US)

(21) Appl. No.: 11/644,293

(22) Filed: Dec. 21, 2006

Related U.S. Application Data
(63) Continuation of application No. PCT/US06/62435, filed on Dec. 20, 2006.
(60) Provisional application No. 60/755,412, filed on Dec. 31, 2005.

(51) Int. Cl. A61K 38/16; A61K 31/7088; A61P 35/00; C07K 14/00; C12N 1/00; C12N 5/06; G01N 33/00; C12Q 1/68; C12N 15/63; C07K 16/18; C07H 21/00; A61K 38/02

(52) U.S. Cl. 514/13; 435/243; 435/320.1; 435/325; 435/6; 436/86; 514/2; 514/44; 514/789; 530/326; 530/387.9; 536/23.1

(57) ABSTRACT

The invention provides COP1 molecules and modulators of COP1 activity and methods of using them, including diagnostic and therapeutic uses thereof. Such molecules can be useful for detecting DNA damage and modulating the response to DNA damage and p53 activity in subjects. The invention also provides reagents and kits for use in screening for test compounds that can modulate COP1 activity.

### RELATED APPLICATION

This application claims priority to and the benefit of U.S. Provisional Application Ser. No. 60/755,412, filed 31 Dec. 2005, the specification of which is incorporated herein in its entirety.

### FIELD OF THE INVENTION

The invention is in the field of COP1 and diagnostics and therapeutics for DNA damage.

### BACKGROUND OF THE INVENTION

Genomic integrity is a prerequisite in mammals to maintain cellular and tissue homeostasis. As such, a specialized group of proteins have been wired into a circuit designed to defend against potential perturbations of the genome. Ataxia Telangiectasia (A-T) is a rare autosomal recessive disease in which affected individuals harbor severe genomic instability at the cellular level¹. Mutations in the Ataxia-telangiectasia mutated protein kinase (ATM) are associated with A-T and implicate ATM as an important component of a DNA-damage response network configured to maintain genomic integrity². ATM functions in part by phosphorylating key substrates including p53, BRCA1, and Chk2. Phosphorylation of ATM substrates are detected post-DNA damage and at the early stages of tumor development⁹.

The tumor suppressor, p53, is involved in the DNA damage activation pathway. Mice null for p53 typically develop lymphomas and sarcomas displaying aneuploidy¹⁰, whereas mice with a mutant p53 capable of only cell cycle arrest form tumors with a diploid chromosome number¹. p53 exerts its tumor suppressor properties by functioning as a stress-activated transcription factor that induces the expression of a cadre of genes whose products are implicated in cell cycle arrest, senescence, or apoptosis. Delaying proliferation of somatic cells with base pair mutations permits the cell to repair such mismatches before duplication of its own genome or cell division. ATM directly phosphorylates p53 at the N-terminus¹² on S15 which increases the transactivation activity of p53 target genes by promoting recruitment of the transcriptional coactivator p300¹³. In addition, ATM phosphorylates MDM2 and MDMX which reduces their ability to negatively regulate p53¹⁴⁻¹⁶. p53 is negatively regulated by E3 ubiquitin ligases⁵, including COP1 which is overexpressed in breast and ovarian cancers^{3,4}.

### SUMMARY OF THE INVENTION

In one aspect, the invention provides a method of detecting DNA damage in a cell that includes an ATM polypeptide by detecting a COP1 polypeptide in the cell, wherein a phosphorylation of the COP1 polypeptide by the ATM polypeptide, or a reduction in the level of the COP1 polypeptide, relative to a control is indicative of DNA damage.

The ATM polypeptide may be a human ATM polypeptide and/or the COP1 polypeptide may be a human COP1 polypeptide. The phosphorylation may be a serine phosphorylation. The COP1 polypeptide may be detected using an antibody that specifically binds a COP1 polypeptide, e.g., a peptide including an amino acid sequence substantially identical to amino acid residues 377-400 of a human COP1 polypeptide, or including an amino acid residue homologous to serine 387 of a human COP1 polypeptide. The peptide may be phosphorylated on a phosphorylatable amino acid residue that is homologous to serine 387 of a human COP1 polypeptide.

The method may further include detecting the ATM polypeptide, where binding of the COP1 molecule to the ATM molecule is indicative of DNA damage, and/or may further include detecting activation of COP1 E3-ligase activity, activation of COP1 auto-ubiquitination, disruption of a p53-COP1 complex, reduction of COP1-dependent p53 ubiquitination, increase in the cytoplasmic-nuclear ratio of COP1, turnover of COP1 polypeptide, or degradation of COP1 polypeptide, and/or may further include detecting a p53 molecule in the cell, where an increase in the expression level of the p53 molecule, or an increase in a p53 activity, relative to a control is indicative of DNA damage. The p53 molecule may be a human p53 molecule and/or a wild type p53 molecule. The p53 molecule may be a p53 polypeptide. The p53 polypeptide may be detected using an antibody that specifically binds the p53 polypeptide. The p53 activity may be activation of p53-dependent transactivation, activation of p53-induced apoptosis, activation of a p21 molecule, induction of a p21 promoter, increase in p21 mRNA levels, or induction of a PUMA promoter.

The DNA damage may be caused by radiation (e.g., ionizing radiation or ultraviolet radiation, or radiation therapy) or by a chemical compound (e.g., an alkylating agent such as a chemotherapeutic agent). The cell (e.g., a cancer cell such as a breast cancer, ovarian cancer, colon cancer, lung cancer, or transitional cell cancer cell) may have or may be at risk for DNA damage. The cancer cell may be obtained from a subject (e.g., a human) undergoing a cancer therapy or having been exposed to radiation or a chemical compound, which exposure can result in DNA damage. The cancer therapy may be known to cause or may be suspected of causing DNA damage.

In an alternative aspect, the invention provides a method of enhancing a response to DNA damage in a cell, by exposing the cell to a compound that enhances degradation of a COP1 polypeptide. The compound may include an ATM molecule, such as an activated ATM polypeptide. The compound may enhance the binding of the COP1 polypeptide to an ATM polypeptide or enhance the phosphorylation of the COP1 polypeptide by an ATM polypeptide.

In an alternative aspect, the invention provides a method of enhancing the interaction of a COP1 polypeptide with an ATM polypeptide, by contacting the COP1 polypeptide and the ATM polypeptide with a compound that enhances the binding of the COP1 polypeptide to the ATM polypeptide.

In alternative embodiments, the method can further comprise the steps of determining whether the binding resulted in the degradation of the COP1 polypeptide, activation of COP1 E3-ligase activity, activation of COP1 auto-ubiquitination, disruption of a COP1-p53 complex, reduction of COP1-dependent p53 ubiquitination, increase in the cytoplasmic/nuclear ratio of COP1 polypeptides, or increase in the expression levels of p53 molecules. The contacting may enhance a response to DNA damage in a cell, such as a cell having or at risk for DNA damage (e.g., an A-T cell or a cancer cell). The response to DNA damage may include cell apoptosis or p53 activation. The p53 activation may be activation of p53-dependent transactivation, activation of p53-induced apoptosis, activation of a p21 molecule, induction of a p21 promoter, or induction of a PUMA promoter. The COP1 polypeptide may be a human COP1 polypeptide and/or the ATM polypeptide may be a human ATM polypeptide.

The present invention contemplates compounds that can: enhance the phosphorylation of an amino acid residue homologous to serine 387 of a human COP1 polypeptide; and/or enhance the phosphorylation of serine 387 of a human COP1 polypeptide.

The present invention also provides a COP1 polypeptide or fragment thereof including a polypeptide with a residue homologous to serine 387 of a human COP1 polypeptide; a polypeptide including a sequence substantially identical to amino acid residues 377-400 of a human COP1 polypeptide; a polypeptide having a phosphorylation on an amino acid residue (e.g., one that is homologous to serine 387 of a human COP1 polypeptide) that is capable of being phosphorylated; a COP1 polypeptide comprising a substitution of threonine, glutamate or aspartate for serine at a residue homologous to serine 387 of a human COP1 polypeptide; and a COP1 mimetic compounds. In one embodiment, the polypeptide is comprises a COP1 polypeptide sequence comprising a residue change at S387 to another residue.

In an alternative aspect, the invention provides a method of identifying a compound that enhances a response to DNA damage in a cell comprising an ATM molecule, by incubating a COP1 polypeptide in the presence or absence of a test compound under a condition suitable for promoting DNA damage in the cell and determining whether degradation of the COP1 polypeptide is enhanced in the presence of the test compound, where a compound that enhances the degradation of the COP1 polypeptide is a compound that enhances a response to DNA damage. The determining may be done relative to a control. The ATM molecule may be capable of phosphorylating the COP1 polypeptide.

In alternative embodiments, the method further includes the option of determining whether activation of COP1 E3-ligase activity, activation of COP1 auto-ubiquitination, disruption of a COP1-p53 complex, reduction of COP1-dependent p53 ubiquitination, increase in the cytoplasmic/nuclear ratio of COP1 polypeptides, increase in p53 activity, or increase in the expression levels of p53 molecule are enhanced by the compound, where such enhancing indicates that the compound is a compound that enhances a response to DNA damage. The p53 activity may be activation of p53-dependent transactivation, activation of p53-induced apoptosis, activation of a p21 molecule, induction of a p21 promoter, or induction of a PUMA promoter. The condition suitable for promoting DNA damage may be radiation. The response to DNA damage may include cell apoptosis or p53 activation.

In an alternative aspect, the invention provides a method for identifying a compound that enhances the interaction of a COP1 polypeptide with an ATM polypeptide, by incubating a COP1 polypeptide with an ATM polypeptide in the presence or absence of a test compound and determining whether the test compound increases or stabilizes the binding of the COP1 polypeptide to the ATM polypeptide, where a test compound that increases or stabilizes the binding of the COP1 polypeptide to the ATM polypeptide is a compound that enhances the interaction of a COP1 polypeptide with an ATM polypeptide.

In alternative aspects, the invention provides an isolated peptide consisting essentially of an amino acid sequence substantially identical to or homologous to amino acid residues 377-400 of human COP1, or an isolated peptide consisting essentially of an amino acid sequence as shown in FIG. 6. In alternative embodiments, the peptide may include a substitution (e.g., an aspartate or glutamate substitution) of a serine in an SQ motif.

In alternative aspects, the invention provides an isolated or recombinant phosphorylated COP1 peptide that includes a phosphorylation homologous to S387 of a human COP1 polypeptide, or an isolated or recombinant COP1 peptide that includes an aspartate or glutamate substitution at an amino acid residue homologous to S387 of a human COP1 polypeptide, or a COP1 mimetic compound.

In alternative embodiments, the invention provides a nucleic acid molecule that encodes the peptide or the mimetic according to the invention. The nucleic acid molecule may be in a vector that is operably linked to a promoter. The vector may further include an ATM nucleic acid molecule operably linked to a promoter. The vector may be in a host cell.

In alternative aspects, the invention provides an antibody or other reagent that specifically binds an amino acid sequence that is phosphorylated on an amino acid residue homologous to serine 387 of human COP1. In alternative embodiments, the invention provides a nucleic acid molecule that encodes the antibody; a vector including the nucleic acid molecule, operably linked to a promoter; a host cell that includes the vector; and/or a kit including the antibody, together with instructions for detecting a phosphorylated COP1 molecule in a cell.

In alternative aspects, the invention provides a pharmaceutical compositions for modulating the response to DNA damage or p53 activity in a subject, including such compositions comprising polypeptides, peptides or mimetics as described herein or nucleic acid encoding the polypeptide or peptide sequences.

In alternative aspects, the invention provides a mammalian cell including a recombinant nucleic acid molecule encoding a COP1 molecule and a recombinant nucleic acid molecule encoding an ATM molecule. The mammalian cell may further include a recombinant nucleic acid molecule encoding a p53 molecule. The ATM molecule may be activated and/or the COP1 molecule may be constitutively phosphorylated or unphosphorylated (e.g., due to mutation at S387).

The invention also provides methods of treating DNA damage or cancer in a subject in need thereof, the method comprising administering to the subject a therapeutically effective amount of a peptide or the mimetic of the invention. Additionally, the invention provides a method of treating DNA damage or cancer in a subject in need thereof, the method comprising administering to the subject a therapeutically effective amount of a compound or a nucleic acid encoding a polypeptide, wherein the compound or polypeptide enhances the binding of a COP1 polypeptide to an ATM polypeptide or enhances the phosphorylation of said COP1 polypeptide by an ATM polypeptide. According to one embodiment, the compound or polypeptide specifically binds COP1. According to another embodiment, the compound or polypeptide specifically binds ATM. According yet another embodiment, the compound or polypeptide specifically binds unphosphorylated COP1. According to yet another embodiment, the compound is administered in an amount effective to increase the phosphorylation of a COP1 polypeptide in the DNA damaged cells or cancer.

The present invention provides compounds identified by the methods of this invention. Additionally, the invention provides use of the peptides, mimetics, nucleic acids, and compounds of this invention in the preparation of a medicament for treating DNA damage or cancer in a subject.

### BRIEF DESCRIPTION OF THE DRAWINGS

FIGS. 1A-E demonstrate that COP1 is turned over post-IR and is dependent upon S387. A, COP1 steady-state levels are reduced upon exposure to IR. B, COP1 mRNA levels are not reduced, but increased upon exposure to IR. C, COP1 is turned over post-IR as assessed by cyclohexamide pulse-chase. D, Potential SQ motifs upon COP1. E, COP1-S387A is resistant to degradation upon exposure to IR.

FIGS. 2A-K ATM negatively regulates COP1. A, Endogenous ATM can phosphorylate COP1 GST-peptides containing S387 in vitro. B, ATM phosphorylates S387 in vivo. C, COP1 interacts with ATM in vivo and is enhanced following DNA damage. D, COP1 reduction in steady-state levels is ATM-dependent. E, Phosphorylation of COP1 at S387 post-IR is dependent upon kinase activity of ATM. F, Exogenous ATM promotes a reduction in COP1 steady-state levels in Saos-2 cells. G, ATM-induced reduction of COP1 is mediated by the 26S proteasome. H, ATM promotes ubiquitination of COP1 in a kinase-dependent manner. I, COP1-C136/139S RING mutant is resistant to ATM-induced degradation. J, Phosphate-mimetic at S387 promotes auto-ubiquitination of COP1 in vitro. K, COP1-S387D displays an enhanced turnover capability.

FIG. 3 ATM promotes COP1 localisation to cytoplasm. A, COP1 is localised to cytoplasm in an ATM-dependent manner following DNA damage. B, COP1-S387D is predominantly localised to the cytoplasmic compartment.

FIGS. 4A-I COP1 negative regulation of p53 is attenuated by ATM modification at S387. A,B COP1 interaction with p53 is dampened upon exposure to DNA damage. C, COP1-S387D is less effective at binding p53. D, p53 displays a reduced ability to bind COP1-S387D in vitro. E, Ubiquitination of p53 by COP1 in vitro is inhibited by a S387D mutation. F. COP1-S387D is less effective at degrading p53. G, COP1-S387D is less potent at inhibiting p53-dependent transactivation. H, COP1-S387D is incapable of negatively regulating p53 tumor suppressor function. I, Model of ATM DNA damage response pathway with respect to the p53 axis.

FIG. 5 S387 on full-length COP1 is the major site for ATM phosphorylation in vitro.

FIG. 6 COP1 SQ3 at S387 is highly conserved in mammalian orthologs.

FIG. 7 ELISA characterization of pS387 COP1 antibody. 1 ng of each peptide was coated on ELISA plate with varying concentration of pS387 antibody. HRP secondary and ECL detected antibody binding to peptide.

FIG. 8 pS387 antibody characterization with in vitro kinase assay. FLAG-ATM and GST-COP1 or GST-COP1-S387A was incubated with or without inhibitors as indicated.

FIG. 9 Steady-state levels of endogenous COP1 are reduced upon co-transfection of ATM in Saos-2. 10 ug of ATM or ATM-KD were transfected for 24 hours and levels assessed by western blotting.

FIG. 10 Imperial stain of SDS-PAGE from HEK293T purified FLAG-COP1-WT, FLAG-COP1-S387D, and FLAG-COP1-RINGmt.

FIG. 11 COP1 is localised to the cytoplasm following IR treatment. Immunofluorescence of transfected FLAG-COP1 in H1299 cells following treatment with 10 Gy IR reveals a significant proportion of COP1 localised to the cytoplasm at the expense of the nuclear COP1 fraction. Immunofluorescence was carried by methanol fixation and biotin-streptavidin amplification method with detection using the Cy5 filter cube on a Zeiss Axiovert 200M microscope. Vectashield (Vector Laboratories) with DAPI was used for staining DNA.

### DETAILED DESCRIPTION OF THE INVENTION

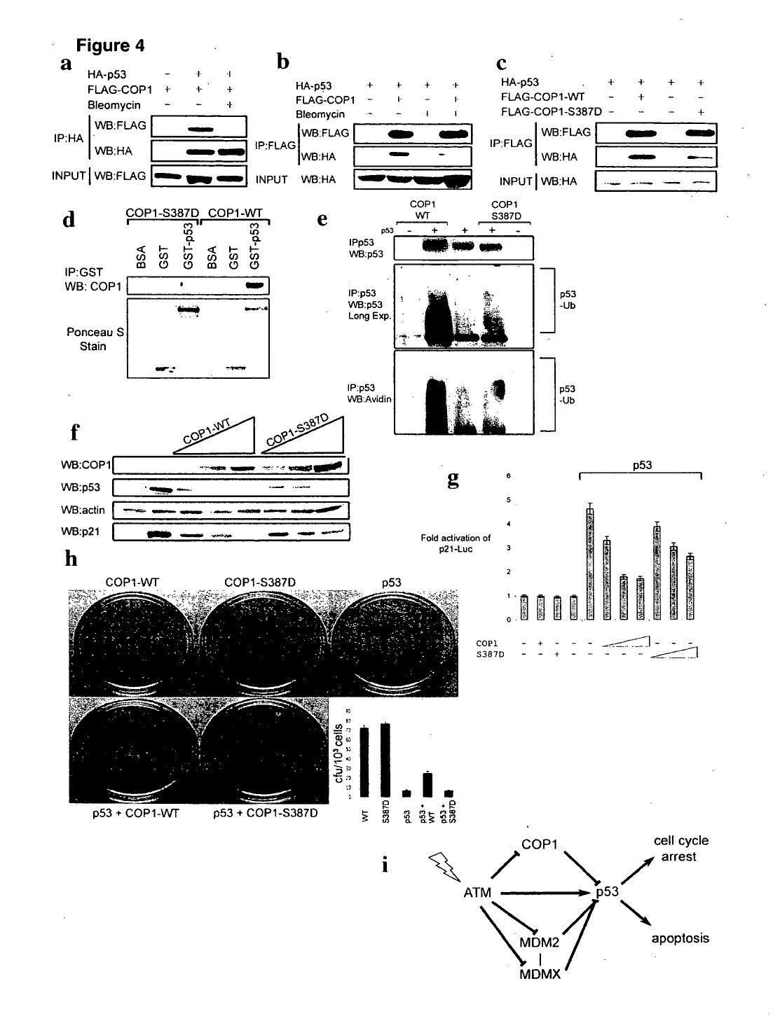

The invention in part demonstrates that ATM interacts with COP1 following DNA damage or changes in chromatin structure and phosphorylates COP 1. This covalent modification by ATM disrupts the COP1-p53 complex and inhibits the ubiquitination, degradation, and negative regulation of p53 tumour suppressor function by COP1. Furthermore, phosphorylation of COP1 engages an auto-E3 degradation mechanism, thereby switching COP1 from trans- to cis-ubiquitin ligase activity, which is the first example of activation of a RING-E3 ligase by phosphorylation. Phosphorylation of COP1 also increases the cytoplasmic/nuclear localisation ratio of COP1. Thus, phosphorylation of COP1 by ATM, or degradation of COP1, leads to a reduced capacity of COP1 to negatively regulate p53-dependent tumour suppressor function. Without being bound to a particular hypothesis, phosphorylation of COP1 by ATM, or degradation of COP1, may provide further explanation for the significantly delayed response to genomic abnormalities in A-T patients, with respect to the p53 pathway (FIG. 4I).

Accordingly, phosphorylation of COP1 by ATM, or degradation of COP1, may be used to detect DNA damage.

Various alternative embodiments and examples of the invention are described herein. These embodiments and examples are illustrative and should not be construed as limiting the scope of the invention.

DNA Damage

By “DNA damage” is meant injury to DNA that affects the normal function of the DNA by causing covalent modification of the DNA or causing it to deviate from its normal double-helical conformation. DNA damage includes structural distortions which interfere with replication and transcription, as well as point mutations which can disrupt base pairs and can change the DNA sequence. In general, when a cell incurs DNA damage, the cell cycle is arrested at one of two checkpoints (G1/S or G2/M). The cell cycle arrest can lead to the activation of DNA repair processes (in the case of relatively minor DNA damage), or result in the induction of apoptosis (in the case of catastrophic DNA damage).

DNA damage can be caused spontaneously by endogenous processes such as oxidation of bases and generation of DNA strand interruptions by reactive oxygen and free radicals produced from normal metabolism, methylation of bases, depurination (e.g., due to the reaction of DNA in water), depyrimidination, mismatch of bases by DNA polymerases during DNA replication, etc. DNA damage can also be caused by environmental insults such as radiation (e.g., ultraviolet radiation, x-rays, gamma rays, ionizing radiation), natural toxins (e.g., plant toxins), synthetic toxins, drugs (e.g., cancer chemotherapy, radiation therapy), alkylating agents, etc.

DNA damage can lead to or result from a variety of disorders, including hereditary genetic disorders and disorders as a result of exposure to environmental insults. Disorders in which DNA damage is a complicating factor include Ataxia telangiectasia (A-T), also called Louis-Bar syndrome or cerebello-oculocutaneous telangiectasia, Xeroderma pigmentosa, Cockayne's syndrome, trichothiodystrophy, Fanconi's anaemia, Bloom's syndrome, and Alzheimer's disease. DNA damage can also occur as a result of smoking, leading to for example, heart disease, or as a result of therapies for other diseases, such as cancers.

Cancers

By a “cancer”, “neoplasm”, “neoplasia”, “carcinoma”, or “tumor” is meant any unwanted growth of cells serving no physiological function. In general, a cell of a neoplasm or cancer e.g., a neoplastic cell, has been released from normal cell division control, i.e., a cell whose growth or proliferation is not regulated by the ordinary biochemical and physical influences in the cellular environment, and exhibits characteristics of unregulated growth, local tissue invasion, metastasis, etc. Generally, a neoplastic cell proliferates to form a clone of cells which are either benign or malignant. The term cancer or neoplasm therefore includes cell growths that are technically benign but which carry the risk of becoming malignant. By “malignancy” is meant an abnormal growth or proliferation of any cell type or tissue. Malignant cells or tissue may inhibit anaplasia or loss of differentiation/orientation, when compared to a normal cell or tissue of the same type, and may exhibit invasion and metastasis capabilites.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to mestastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) e.g., acute lymphoblastic leukemia, chronic lymphocytic leukemia, acute monocytic leukemia, acute promyelocytic leukemia, chronic myelocytic leukemia,etc., or polycythemia vera or erythremia (various blood cell products, but with red cells predominating); or lymphomas, which may be solid tumors and which develop in the glands or nodes of the lymphatic system, and which may include Hodgkin or Non-Hodgkin lymphomas, Burkitt's lymphoma, etc. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body, that is different from the primary site, and cancers according to the invention include primary cancers, as well as cancers that have metastasized.

Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas e.g., malignant melanoma. Lymphomas may arise in the lymph nodes associated with the head, neck and chest, as well as in the abdominal lymph nodes or in the axillary or inguinal lymph nodes. Identification and classification of types and stages of cancers may be performed by using for example information provided by the Surveillance, Epidemiology, and End Results (SEER) Program of the National Cancer Institute (http://seer.cancer.gov/publicdata/access.html), which is an authoritative source of information on cancer incidence and survival in the United States and is recognized around the world. The incidence and survival data of the SEER Program may be used to access standard survival for a particular cancer site and stage. For example, to ensure an optimal comparison group, specific criteria may be selected from the database, including date of diagnosis and exact stage. Identification of cancers may also be performed by using for example information provided in diagnostic manuals such as The Merck Manual of Diagnosis and Therapy, 17^{th} edition, M. H. Beers and R. Barkow, eds., John Wiley and Sons, 1999.

Examples of cancers or neoplasms may also include, without limitation, transformed and immortalized cells, solid tumors, myeloproliferative diseases, blastomas, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, serous adenocarcinoma, endometrioid adenocarcinoma, clear cell adenocarcinoma, mucinous adenocarcinoma, Brenner tumor, teratoma, dysgerminoma, choriocarcinoma, fibroma, granulosa cell tumor, Sertoli-Leydig cell tumor, undifferentiated ovarian carcinoma, salivary gland carcinoma, kidney or renal cancer, prostate cancer, vulval cancer, thyroid cancer, hepatic carcinoma, anal carcinoma, penile carcinoma, cancer of the head and/or neck, Ewing's sarcoma, hemangiosarcoma, Kaposi's sarcoma, liposarcoma, peripheral neuroepithelioma, synovial sarcoma, Hodgkin's disease, etc. as known in the art.

Polypeptides, Nucleic Acid Molecules, And Compounds

Compounds according to the invention include, without limitation, COP1 or ATM nucleic acid molecules, polypeptides and/or isoforms, variants, mimetics, homologs, analogs, or fragments thereof. In some embodiments, the invention also makes use of p53, p21, and other nucleic acid molecules, polypeptides and/or isoforms, variants, mimetics, homologs, analogs, or fragments thereof.

COP1 Compounds

A “COP1 molecule” as used herein refers to a molecule substantially identical to: a COP1 polypeptide; a nucleic acid molecule encoding a COP1 polypeptide; a COP1 nucleic acid molecule; as well as isoforms, variants, mimetics, homologs, analogs, or fragments thereof. A COP1 molecule may include, without limitation, polypeptide or nucleic acid molecules containing sequences substantially identical to those set forth in Accession Nos. AF508940 (human), AAH82804 (mouse), NP_{—}036061 (mouse), NP_{—}001001740 (human, isoform d24), NP_{—}071902 (human, isoform a), XP_{—}468011 (Oryza sativa), XP_{—}468010 (Oryza sativa), XP_{—}463866 (Oryza sativa), AAM34692 (human), AAH39723 (human), BAB45239 (human), P_ABG08243 (human), AAD51094 (mouse), AAN86553 (Brassica rapa subsp. Pekinensis), CAA98718 (Saccharomyces cerevisiae), CAA04168 (Arabidopsis thaliana), XM_{—}477896 (Oryza sativa), XM_{—}479164 (Oryza sativa), BK000438 (human), AF508940 (human), AF151110 (mouse), L24437 (Arabidopsis thaliana), P_AAY60008 (human), P_ABJ19398 (human), P_ABB11576(human), P_ABG95247 (human), P_AAW74797 (human), P_ABP69180 (human), P_AAB92798 (human), XP_{—}064815 (human), and/or P_AAG02591 (human), as well as isoforms, variants, mimetics, homologs, analogs, or fragments thereof. A COP1 molecule may be a molecule as described in Bianchi et al.,⁶ Wang et al.,⁷ or Yi et al.¹⁹

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any value from 10% to 99%, or more generally at least 10%, 20%, 30%, 40%, 50, 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program¹⁸ or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications. Substantially identical sequences include homologous sequences, such as COP1 related sequences from non-human species as described herein or known in the art.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al.⁸, which is hereby incorporated by reference.

In some embodiments, a human COP1 molecule includes a sequence as set forth in Accession No. AF508940, or a fragment thereof.

In some embodiments, a human COP1 nucleic acid molecule includes the following sequence, or a fragment thereof:

```
      1 atgtctggta gccgccaggc cgggtcgggc tccgctggga caagccccgg gtcctcggcg
    61 gcctcctcgg tgacttccgc ctcctcgtct ttatcctctt ccccgtcgcc gccttccgtg
  121 gcggtttcgg cggcagcgct ggtgtccggc ggggtggccc aggccgccgg ctcgggcggc
  181 ctcgggggcc cggtgcggcc tgtgttggtg gcgcccgccg tatcgggtag cggcggcggg
  241 gcggtgtcca cgggcctgtc ccggcacagc tgcgcggcca ggcccagcgc cggcgtagga
  301 ggcagcagct ccagcctagg cagcggcagc aggaagcgac ctctcctcgc ccccctctgc
  361 aacgggctca tcaactccta cgaggacaaa agcaacgact tcgtatgccc catctgcttt
  421 gatatgattg aagaagcata catgacaaaa tgtggccaca gcttttgcta caagtgtatt
  481 catcagagtt tggaggacaa taatagatgt cccaagtgta actatgttgt ggacaatatt
  541 gaccatctgt atcctaattt cttggtgaat gaactcattc ttaaacagaa gcaaagattt
  601 gaggaaaaga ggttcaaatt ggaccactca gtgagtagca ccaatggcca caggtggcag
  661 atatttcaag attggttggg aactgaccaa gataaccttg atttggccaa tgtcaatctt
  721 atgttggagt tactagtgca gaagaagaaa caactggaag cagaatcaca tgcagcccaa
  781 ctacagattc ttatggaatt cctcaaggtt gcaagaagaa ataagagaga gcaactggaa
  841 cagatccaga aggagctaag tgttttggaa gaggatatta agagagtgga agaaatgagt
  901 ggcttatact ctcctgtcag tgaggatagc acagtgcctc aatttgaagc tccttctcca
  961 tcacacagta gtattattga ttccacagaa tacagccaac ctccaggttt cagtggcagt
1021 tctcagacaa agaaacagcc ttggtataat agcacgttag catcaagacg aaaacgactt
1081 actgctcatt ttgaagactt ggagcagtgt tacttttcta caaggatgtc tcgtatctca
1141 gatgacagtc gaactgcaag ccagttggat gaatttcagg aatgcttgtc caagtttact
1201 cgatataatt cagtacgacc tttagccaca ttgtcatatg ctagtgatct ctataatggt
1261 tccagtatag tctctagtat tgaatttgac cgggattgtg actattttgc gattgctgga
1321 gttacaaaga agattaaagt ctatgaatat gacactgtca tccaggatgc agtggatatt
1381 cattaccctg agaatgaaat gacctgcaat tcgaaaatca gctgtatcag ttggagtagt
1441 taccataaga acctgttagc tagcagtgat tatgaaggca ctgttatttt atgggatgga
1501 ttcacaggac agaggtcaaa ggtctatcag gagcatgaga agaggtgttg gagtgttgac
1561 tttaatttga tggatcctaa actcttggct tcaggttctg atgatgcaaa agtgaagctg
1621 tggtctacca atctagacaa ctcagtggca agcattgagg caaaggctaa tgtgtgctgt
1681 gttaaattca gcccctcttc cagataccat ttggctttcg gctgtgcaga tcactgtgtc
1741 cactactatg atcttcgtaa cactaaacag ccaatcatgg tattcaaagg acaccgtaaa
1801 gcagtctctt atgcaaagtt tgtgagtggt gaggaaattg tctctgcctc aacagacagt
1861 cagctaaaac tgtggaatgt agggaaacca tactgcctac gttccttcaa gggtcatatc
1921 aatgaaaaaa actttgtagg cctggcttcc aatggagatt atatagcttg tggaagtgaa
1981 aataactctc tctacctgta ctataaagga ctttctaaga ctttgctaac ttttaagttt
2041 gatacagtca aaagtgttct cgacaaagac cgaaaagaag atgatacaaa tgaatttgtt
2101 agtgctgtgt gctggagggc actaccagat ggggagtcca atgtgctgat tgctgctaac
2161 agtcagggta caattaaggt gctagaattg gtatga
```

In some embodiments, a human COP1 polypeptide includes the following sequence, or a fragment thereof:

MSGSRQAGSGSAGTSPGSSAASSVTSASSSLSSSPSPPSVAVSAAALVSG
GVAQAAGSGGLGGPVPPVLVAPAVSGSGGGAVSTGLSRHSCAARPSAGVG
GSSSSLGSGSRKRPLLAPLCNGLINSYEDKSNDFVCPICFDMIEEAYMTK
CGHSFCYKCIHQSLEDNNRCPKCNYVVDNIDHLYPNFLVNELILKQKQRF
EEKRFKLDHSVSSTNGHRWQIFQDWLGTDQDNLDLANVNLMLELLVQKKK
QLEAESHAAQLQILMEFLKVARRNKREQLEQIQKELSVLEEDIKRVEEMS
GLYSPVSEDSTVPQFEAPSPSHSSIIDSTEYSQPPGFSGSSQTKKQPWYN
STLASRRKRLTAHFEDLEQCYFSTRMSRISDDSRTASQLDEFQECLSKFT
RYNSVRPLATLSYASDLYNGSSIVSSIEFDRDCDYFAIAGVTKKIKVYEY
DTVIQDAVDIHYPENEMTCNSKISCISWSSYHKNLLASSDYEGTVILWDG
FTGQRSKVYQEHEKRCWSVDFNLMDPKLIASGSDDAKVKLWSTNLDNSVA
SIEAKANVCCVKFSPSSRYHLAFGCADHCVHYYDLRNTKQPIMVFKGHRK
AVSYAKFVSGEEIVSASTDSQLKLWNVGKPYCLRSFKGHINEKNFVGLAS
NGDYTACGSENNSLYLYYKGLSKTLLTFKFDTVKSVLDKDRKEDDTNEFV
SAVCWRALPDGESNVLIAANSQGTIKVLELV
(Serine 387 underlined)

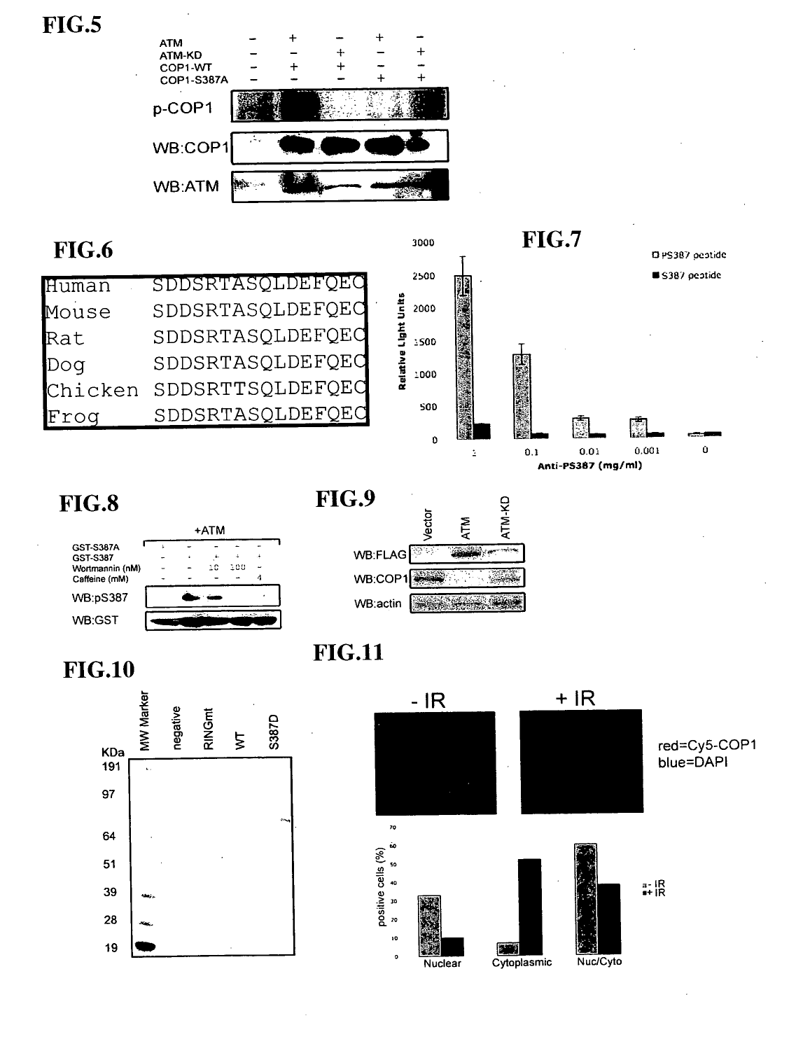

In some embodiments, a COP1 polypeptide may include amino acids 377-400 of a human COP1 polypeptide, or a fragment thereof. In some embodiments, a COP1 peptide may include, without limitation, the following sequences: DSRTASQLDEFC, SRTASQ, RTASQ, TASQ, ASQ, SQLDE, SQLD, SQL, ASQL, TASQLD, RTASQLDE, or SRTASQLDEF, or sequences substantially identical thereto. In some embodiments, a COP1 peptide may include, without limitation, an amino acid sequence of any value between 3 to 20 amino acids, such as 5 amino acids, 7 amino acids, 10 amino acids, 12 amino acids, or 15 amino acids, where the amino acid sequence includes a sequence homologous to S387 of a human COP1 polypeptide. In some embodiments, a COP1 polypeptide may be capable of directly binding an ATM polypeptide, or being phosphorylated by an ATM polypeptide. In some embodiments, a COP1 polypeptide may include a molecule substantially identical to the amino acid sequences set forth herein that are capable of being phosphorylated by ATM. In some embodiments, a COP1 polypeptide may be a polypeptide that is phosphorylated on, or is capable of being phosphorylated on, a residue homologous to S387 of a human COP1 polypeptide. Sequences homologous to S387 of a human COP1 polypeptide are described in, for example, FIG. 6. In some embodiments, a COP1 molecule includes a COP1 polypeptide that is constitutively phosphorylated at an amino acid residue homologous to serine 387 of a human COP1 polypeptide. In some embodiments, a COP1 molecule includes a COP1 polypeptide that includes a substitution of serine 387 of a human COP1 polypeptide for a phosphorylatable amino acid residue.

A “phosphorylated” COP1 protein or polypeptide is post-translationally modified on any amino acid residue capable of being phosphorylated in vivo (e.g, serine, threonine, tyrosine, histidine). In the context of the present invention, a phosphorylated COP1 polypeptide is generally phosphorylated on an amino acid residue homologous to Serine 387 (S387) of a human COP1 polypeptide. An “unphosphorylated” COP1 polypeptide may be incapable of being phosphorylated on an amino acid residue capable of being phosphorylated in vivo, for example, by mutation of that residue to an amino acid that is not capable of being phosphorylated. A mutation of a serine to an alanine in a polypeptide sequence, for example, results in a protein that is not capable of being phosphorylated at that particular position in the polypeptide sequence. A COP1 polypeptide that possesses an alanine at a position homologous to position 387 of a human COP1 polypeptide instead of a serine is such an “unphosphorylated” COP1 polypeptide. An unphosphorylated COP1 polypeptide may also be a polypeptide that is capable of being phosphorylated in vivo, for example on S387, but is not phosphorylated due to, for example, the presence of an inhibitor, for example, a kinase inhibitor such as an ATM kinase inhibitor; due to an antibody that interferes with the phosphorylation site; or due to the activity of a phosphatase. A “constitutively phosphorylated” COP1 polypeptide is a polypeptide that possesses a mutation at an amino acid residue homologous to, for example, S387 of a human COP1 polypeptide, that is capable of being phosphorylated in vivo, where the mutation mimics phosphorylation at that residue, and the resultant polypeptide possesses the biological activity of a phosphorylated polypeptide. Generally, mutation of a phosphorylatable residue to a glutamic acid or aspartic acid residue results in constitutive phosphorylation. In some embodiments, a phosphorylated or constitutively phosphorylated COP1 peptide may have, without limitation, the following sequences: DSRTA(pS/T/Y/D/E)QLDEFC, SRTA(pS/T/Y/D/E)Q, RTA(pS/T/Y/D/E)Q, TA(pS/T/Y/D/E)Q, A(pS/T/Y/D/E)Q, (pS/T/Y/D/E)QLDE, (pS/T/Y/D/E)QLD, (pS/T/Y/D/E)QL, A(pS/T/Y/D/E)QL, TA(pS/T/Y/D/E)QLD, RTA(pS/T/Y/D/E)QLDE, SRTA(pS/T/Y/D/E)QLDEF, where “pS/T/Y/D/E” denotes a serine, threonine, or tyrosine phosphorylation, or the substitution of the phosphorylatable serine for aspartate or glutamate.

In some embodiments, compounds according to the invention include a mimetic of a COP1 polypeptide. A mimetic compound, in general, is based on a COP1 polypeptide, such as an unphosphorylated or a phosphorylated COP1 polypeptide, e.g., a COP1 polypeptide substantially identical to the peptides described herein or a COP1 polypeptide phosphorylated on an amino acid residue homologous to S387 and may include a compound having for example a peptide-like secondary structure. A mimetic compound may result in an enhancement of COP1 activity or selectivity, or may exhibit reduced degradation characteristics for prolongation of the mimetic effect. A mimetic compound may be an agonist or an antagonist (inhibitor). A mimetic compound may include e.g., a cyclic peptide, a peptide analog, a constrained peptide, a scaffold mimetic, a non-peptidic mimetic, a peptide nucleic acid, etc. A mimetic compound may bind an ATM polypeptide with a binding affinity of any value between 0.001 pm to 1 uM, such as at least 500 nM, 100 nM, 10 nM, 1 nM, 500 pM, 100 pM, 10 pM, etc. A mimetic compound may have a molecular weight of less than 2000 Da, e.g., 1500 Da, 1000 Da, or 500 Da. Exemplary COP1 mimetic compounds may include, without limitation, compounds including the following sequences: SRTASQ, RTASQ, TASQ, ASQ, SQLDE, SQLD, SQL, ASQL, TASQLD, RTASQLDE, SRTASQLDEF, including compounds in which the serine in the “SQ” motif is phosphorylated or constitutively phosphorylated, and/or compounds in which the peptide structure or sequence is modified as described herein or known in the art. In some embodiments, a compound according to the invention includes a COP1 mimetic compound that mimics a phosphorylated COP1 molecule or phosphorylated fragment thereof

In some embodiments, compounds according to the invention include antibodies or other reagents (e.g., peptibodies) that specifically bind to COP1, e.g., to phosphorylated COP1 such as a COP1 peptide phosphorylated on an amino acid residue homologous to serine 387 of a human COP1 polypeptide, or to a peptide including the following sequences: SRTASQ, RTASQ, TASQ, ASQ, SQLDE, SQLD, SQL, ASQL, TASQLD, RTASQLDE, SRTASQLDEF. Such antibodies may be for example polyclonal or monoclonal or may be humanized antibodies. Such peptibodies may be peptides that specifically bind COP1 fused to an Fc portion of an immunoglobulin antibody (e.g., IgG1-4 ). An antibody or other reagent that “specifically binds” an antigen when it recognises and binds the antigen, but does not substantially recognise and bind other molecules in a sample. For example, a COP1 antibody specifically binds a COP1 molecule, but does not substantially bind any other molecule such as those present in a cancer cell or tissue, or in a cell having DNA damage. In some embodiments, a COP1 antibody or other reagent may specifically bind a human COP1 molecule and may not specifically bind COP1 molecules from other species. In some embodiments, a COP1 antibody or other reagent may specifically bind a phosphorylated COP1 molecule and may not specifically bind non-phosphorylated COP1 molecules. In some embodiments, a COP1 antibody or other reagent may specifically bind a phosphorylated COP1 molecule on a specific amino acid residue e.g. S387 of a human COP1 polypeptide and may not specifically bind COP1 molecules phosphorylated on other amino acid residues. An antibody or other reagent that specifically binds an antigen has, for example, an affinity for the antigen which is at least 10, 100, 1000 or 10000 times greater than the affinity of the antibody or reagent for another reference molecule in a sample.

In some embodiments, a COP1 molecule includes a COP1 nucleic acid molecule that encodes a COP1 polypeptide, or encodes an antibody or other reagent that specifically binds to a COP1 polypeptide.

Other Compounds

An “ataxia telangiectasia mutated” or “ATM molecule” as used herein refers to a molecule substantially identical to: an ATM polypeptide; a nucleic acid molecule encoding an ATM polypeptide; an ATM nucleic acid molecule; as well as isoforms, fragments, homologs, analogs, or variants thereof. An ATM molecule may include, without limitation, polypeptide or nucleic acid molecules containing sequences substantially identical to those set forth in Accession Nos. Q13315 (human), NM_{—}007499 (mouse), NM_{—}138292 (human), NM_{—}000051 (human), NM_{—}114689 (Arabidopsis thaliana), BC022307 (human), BC061584 (human), XM_{—}777432 (sea urchin), BC007023 (human), XM_{—}680015 (zebrafish), XM_{—}236275 (rat), AAB65827 (human), etc. In some embodiments, the ATM polypeptide is activated and has kinase activity. In general, an ATM polypeptide is capable of phosphorylating a wild type COP1 molecule, or a COP1 molecule that is capable of being phosphorylated on an amino acid residue homologous to serine 387 of a human COP1 polypeptide. ATM kinase activity may be generated for example by DNA damage, e.g., ionizing radiation, or by a chemical compound capable of causing DNA damage. ATM kinase activity may be assessed as described herein or known in the art. In some embodiments, an ATM molecule includes an ATM polypeptide, or fragment thereof, that is capable of binding and/or phosphorylating a COP1 molecule.

“p53” is a potent tumor suppressor protein encoding a 393 amino acid phosphoprotein. p53 is negatively regulated or mutated in many cancers. Absence or inactivation of p53 may contribute to cancer. A wide variety of p53 mutations exist. A “wild type” p53 is p53 found in normal i.e., non-cancerous cells, or p53 that does not have a mutation correlated to a cancer. The p53 status of a sample (e.g., whether the sample includes wild type or mutant p53) may be assessed as for example described in U.S. Pat. No. 6,090,566 issued to Vogelstein et al., or using standard techniques such as described herein or known in the art. A p53 molecule may include, without limitation, polypeptide or nucleic acid molecules containing sequences substantially identical to that set forth in for example Accession No. P04637.

p21 or “WAF1/Cip1” was originally described as a universal inhibitor of cyclin-dependent kinases. It is induced by both p53-dependent and p53-independent mechanisms and has been implicated as an inhibitor of cell proliferation.

Preparation of Polypeptides, Nucleic Acid Molecules, And Test Compounds

It is well known in the art that some modifications and changes can be made in the structure of a polypeptide without substantially altering the biological function of that peptide, to obtain a biologically equivalent polypeptide having for example desired characteristics such as enhanced stability, etc. In one aspect of the invention, compounds of the present invention also extend to biologically equivalent peptides or mimetics that differ from a portion of the sequence of the COP1 polypeptides of the present invention by amino acid or other substitutions that do not affect biological function.

For example, COP1 compounds can be prepared by, for example, replacing, deleting, or inserting an amino acid residue at any position of a COP1 peptide or peptide analog or mimetic, for example, at a position homologous to serine 387 of a human COP1 polypeptide, with other conservative amino acid residues, i.e., residues having similar physical, biological, or chemical properties, e.g., threonine, glutamate or aspartate, in place of serine 387 of a human COP1 polypeptide, or with non-conservative amino acid residues and screening for example for the ability of the compound to bind to an ATM polypeptide, or to be phosphorylated by an activated ATM polypeptide, or to disrupt a p53/COP1 complex. Such compounds may be used in any of the diagnostic, therapeutic, screening, etc. methods of the invention.

As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. In some embodiments, conserved amino acid substitutions refer to the replacement of an amino acid residue homologous to serine 387 of a human COP1 polypeptide with a phosphorylatable amino acid, such as threonine or tyrosine, or with a phosphorylation mimic, such as glutamate or aspartate.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Omithine.

In some embodiments, conserved amino acid substitutions may be made where an amino acid residue is substituted for another having a similar hydrophilicity value (e.g., within a value of plus or minus 2.0, or plus or minus 1.5, or plus or minus 1.0, or plus or minus 0.5), where the following may be an amino acid having a hydropathic index of about −1.6 such as Tyr (−1.3) or Pro (−1.6) are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conservative amino acid substitutions may be made where an amino acid residue is substituted for another having a similar hydropathic index (e.g., within a value of plus or minus 2.0, or plus or minus 1.5, or plus or minus 1.0, or plus or minus 0.5). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conservative amino acid substitutions may be made using publicly available families of similarity matrices²³⁻²⁹. The PAM matrix is based upon counts derived from an evolutionary model, while the Blosum matrix uses counts derived from highly conserved blocks within an alignment. A similarity score of above zero in either of the PAM or Blosum matrices may be used to make conservative amino acid substitutions.

In alternative embodiments, conservative amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al.³⁰ . Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Trp, while non-genetically encoded aromatic amino acids include phenylglycine, 2-napthylalanine, beta-2-thienylalanine, 1,2,3,4-tetrahydro-isoquinoline-3-carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine. It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine. Peptide analogs or mimetics also include amino acids that have been chemically altered, for example, by methylation, by amidation of the C-terminal amino acid by an alkylamine such as ethylamine, ethanolamine, or ethylene diamine, or acylation or methylation of an amino acid side chain (such as acylation of the epsilon amino group of lysine). Peptide analogs or mimetics can also include replacement of the amide linkage in the peptide with a substituted amide (for example, groups of the formula —C(O)—NR, where R is (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl) or isostere of an amide linkage (for example, —CH₂NH—, —CH₂S, —CH₂CH₂—, —CH═CH— (cis and trans), —C(O)CH₂—, —CH(OH)CH₂—, or —CH₂SO—).

The compound can be covalently linked, for example, by polymerisation or conjugation, to form homopolymers or heteropolymers. Spacers and linkers, typically composed of small neutral molecules, such as amino acids that are uncharged under physiological conditions, can be used. Linkages can be achieved in a number of ways. For example, cysteine residues can be added at the peptide termini, and multiple peptides can be covalently bonded by controlled oxidation. Alternatively, heterobifunctional agents, such as disulfide/amide forming agents or thioether/amide forming agents can be used. The compound can also be linked to a another compound that can for example, target cancer cells or cells with DNA damage or inhibit the growth or proliferation of cancer cells or cells with DNA damage, or induce death of cancer cells or cells with DNA damage. The compound can also be constrained, for example, by having cyclic portions.

Peptides or peptide analogs or mimetics can be synthesised by standard chemical techniques, for example, by automated synthesis using solution or solid phase synthesis methodology. Automated peptide synthesisers are commercially available and use techniques well known in the art. Peptides and peptide analogs or mimetics can also be prepared using recombinant DNA technology using standard methods such as those described in, for example, Sambrook, et al.³¹ or Ausubel et al.⁸.

In some embodiments, compounds of the invention include nucleic acid molecules that are substantially identical to COP1 or ATM nucleic acid molecules or fragments thereof, or are complementary to COP1 or ATM nucleic acid molecules or fragments thereof. Such nucleic acid molecules may be used for example as probes or primers in the assays and methods of the invention. A “probe” or “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are known to those skilled in the art. Probes or primers specific for the nucleic acid sequences described herein, or portions thereof, may vary in length by any integer from at least 8 nucleotides to over 500 nucleotides, including any value in between, depending on the purpose for which, and conditions under which, the probe or primer is used. For example, a probe or primer may be 8, 10, 15, 20, or 25 nucleotides in length, or may be at least 30, 40, 50, or 60 nucleotides in length, or may be over 100, 200, 500, or 1000 nucleotides in length. Probes or primers specific for the nucleic acid molecules described herein may have greater than 20-30% sequence identity, or at least 55-75% sequence identity, or at least 75-85% sequence identity, or at least 85-99% sequence identity, or 100% sequence identity to the nucleic acid sequences described herein.

Probes or primers may be derived from genomic DNA or cDNA, for example, by amplification, or from cloned DNA segments, and may contain either genomic DNA or cDNA sequences representing all or a portion of a single gene from a single individual. A probe may have a unique sequence (e.g., 100% identity to a COP1 or ATM nucleic acid molecule) and/or have a known sequence. Probes or primers may be chemically synthesized.

Probes or primers can be detectably-labeled, either radioactively or non-radioactively, by methods that are known to those skilled in the art. Probes or primers can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are known to those skilled in the art.

Nucleic acid molecules may also be antisense molecules, siRNA molecules, or triple helix molecules that may be used for example to reduce expression of the target molecule in a cell.

In some embodiments, test compounds include small organic molecules. A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

In some embodiments of the invention, test compounds include peptides, nucleic acid molecules, mimetics or small molecules, antibodies or other reagents that are capable of mediating COP1/ATM interaction, e.g., phosphorylation or binding.

Candidate or test compounds may be identified from large libraries of both natural products or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla., USA), and PharmaMar, Mass., USA. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to for example enhance COP1/ATM interaction, further fractionation of the positive lead extract is necessary to isolate chemical constituents responsible for the observed effect. Thus, the goal of the extraction, fractionation, and purification process is the careful characterization and identification of a chemical entity within the crude extract having COP1/ATM interaction enhancing activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic, prophylactic, diagnostic, or other value may be subsequently analyzed using for example any animal model for cancer or radiation damage.

Diagnostic, Therapeutic, Prophylactic and/or Screening Uses, Assays, and Reagents Compounds, compositions (e.g., pharmaceutical compositions), and methods according to the invention may be used to diagnose or detect DNA damage, to modulate a response to DNA damage and/or p53 activity in a subject, or to screen or identify test compounds useful for modulating a response to DNA damage and/or p53 in a subject.

As used herein, a subject may be a human, non-human primate, rat, mouse, cow, horse, pig, sheep, goat, dog, cat, fly, worm, etc. The subject may be a clinical patient, a clinical trial volunteer, an experimental animal, etc. The subject may be suspected of having or being at risk for having a cancer or DNA damage, be diagnosed with a cancer or DNA damage, or be a control subject that is confirmed to not have a cancer or DNA damage, or be a subject in which DNA damage or cancer is induced. In one preferred embodiment, the subject is a human.

As discussed herein, DNA damage may be diagnosed or detected by measuring a reduction in COP1 expression levels, where the reduction in expression of COP1 indicates DNA damage, or by measuring COP1 phosphorylation, where the presence of COP1 phosphorylation on an amino acid homologous to serine 387 of a human COP1 polypeptide indicates DNA damage.

By “detecting” it is intended to include determining the presence or absence of a substance or quantifying the amount of a substance. The term thus refers to the use of the compounds, compositions, and methods of the present invention for qualitative and quantitative determinations. In general, the particular technique used for detection is not critical for practice of the invention. For example, “detecting” according to the invention may include detecting: a change in phosphorylation of a COP1 polypeptide, e.g., at an amino acid residue homologous to S387 of a human COP1 polypeptide; the presence or absence of a COP1, ATM, p21, or p53 gene, genome, or nucleic acid molecule or a COP1, ATM, p21, or p53 polypeptide; a mutation in a COP1, ATM, p21, or p53 gene; a change in expression levels of a COP1, ATM, p21, or p53 nucleic acid molecule, e.g., mRNA or polypeptide; a change in a biological function/activity of a COP1 polypeptide (e.g., COP1 ligase activity, p53 turnover, repression of p53-dependent transactivation activity) or a p53 polypeptide (e.g., p53 binding , p53-dependent transactivation, COP1 binding, transactivation of p21, etc.), using methods that are known in the art or described below. In some embodiments, “detecting” may include detecting wild type p53. In some embodiments, “detecting” may include detecting mutant p53. Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, e.g., 3 fold, 5 fold, 7 fold, or more e.g., 50 fold or 100-fold when compared to a control.

“Enhancing” a response, or enhancing interaction e.g., binding or phosphorylation, may include promoting a response or interaction, or increasing an already present response or interaction by any value between 10% and 90%, or between 30% and 60%, or over 100%, when compared to a control. Enhancing may include an increase of any value between 2-fold to 10-fold, inclusive, e.g, 3 fold, 5 fold, 7 fold, or more e.g., 50 fold or 100-fold when compared to a control. For example, “enhancing” according to the invention may include increasing or promoting: a change in phosphorylation of a COP1 polypeptide, e.g., at an amino acid residue homologous to S387 of a human COP1 polypeptide; a change in expression levels of a COP1, ATM, p21, or p53 nucleic acid molecule, e.g., mRNA or polypeptide; a change in a biological function/activity of a COP1 polypeptide (e.g., COP1 ligase activity, p53 turnover, repression of p53-dependent transactivation activity) or a p53 polypeptide (e.g., p53 binding, p53-dependent transactivation, COP1 binding, transactivation of p21, etc.), using methods that are known in the art or described below

By “reduction” in expression is meant a decrease in polypeptide expression of a particular molecule e.g., COP1, relative to a control e.g., relative to the level of expression that is normally produced by cells that do not have substantial DNA damage. Cells which exhibit reduction in expression of a COP1 molecule may also exhibit increased expression of a p53 molecule (e.g., a p53 polypeptide) or increased expression of a p21 molecule (e.g., a p21 mRNA), or increased p53 or p21 activity. Such an increase or decrease may of any value between 10% and 90%, or of any value between 30% and 60%, or over 100%, or may be a change of any value between 2-fold to 10-fold, inclusive, e.g., 3 fold, 5 fold, 7 fold, or more e.g., 50 fold or 100-fold when compared to a control. The exact amount of overexpression or increase, or reduction or decrease, is not critical, as long as the overexpression or reduction is statistically significant.

A “control” includes a sample obtained for use in determining base-line expression or activity. Accordingly, a control sample may be obtained by a number of means including from cells not having DNA damage (as determined by standard techniques); non-cancerous cells or tissue e.g., from cells surrounding a tumor or cancerous cells of a subject; from subjects not having a cancer or a DNA damage disorder; from subjects not suspected of being at risk for a cancer or for DNA damage; or from cells or cell lines derived from such subjects. A control also includes a previously established standard. Accordingly, any test or assay conducted according to the invention may be compared with the established standard and it may not be necessary to obtain a control sample for comparison each time. In an in vitro ubiquitination assay, a control can be a COP1 molecule that has reduced ability to ubiquitinate a p53 molecule (e.g., a molecule that is defective in its ligase domain such as COP1ΔRing). In an in vitro or in vivo kinase assay, a control can be an ATM that is kinase-dead, a COP1 molecule that is known to be phosphorylated, or that is unphosphorylatable, etc.

Reagents according to the invention include compounds as described herein. In some embodiments, the invention encompasses cells, e.g., mammalian cells, that include compounds as described herein. For example, a mammalian cell can be engineered by for example recombinant techniques to include recombinant ATM, recombinant COP1 and/or recombinant p53 molecules. Such mammalian cells may for example be used to screen for test compounds that enhance COP1/ATM interaction (e.g., binding and/or phosphorylation), disrupt p53/COP1 binding or increase p53 or COP1 activity. Such cells could be incubated with a test compound and assayed for changes in cell cycle, ATM kinase activity, p21 expression, p53 induced apoptosis, p53 dependent transactivation, COP1 ligase activity, etc. Reporter based constructs such as p21-Luciferase could be used, with an internal Renilla Luciferase reporter as a background as well as real-time RT-PCR techniques. Such mammalian cells could be engineered in existing cell lines such as for example a p53 wild-type cell line (U2-OS cells), a p53 null cell line (H1299), an ATM null cell line (GM02052 or GM09607) which can be engineered to express COP1 molecules, ATM molecules or p53 molecules. Thus, COP1 or ATM molecules may be provided in A-T cells, cancer cells, tissues, or cell lysates, or may be constructed using recombinant techniques. Cells and/or cell lines may be obtained from commercial sources, for example, ATCC, Manassas, Va., USA.

Assays according to the invention may be carried out in vivo, in vitro, or ex vivo using samples obtained from standard sources and by standard procedures. Microarrays, for example, tissue microarrays may be used. In vivo assays may be performed in suitable animal models for cancer or DNA damage, which may be obtained from, for example, The Jackson Laboratory, Bar Harbor, Me., USA.

In some embodiments, an animal model having defects in ATM, COP1 or p53 expression or activity may be used. A “sample” can be any organ, tissue, cell, or cell extract isolated from a subject, such as a sample isolated from a mammal having a cancer or having DNA damage. For example, a sample can include, without limitation, cells or tissue (e.g., from a biopsy or autopsy) from bone, brain, breast, colon, muscle, nerve, ovary, prostate, retina, skin, skeletal muscle, intestine, testes, heart, liver, kidney, stomach, pancreas, uterus, adrenal gland, tonsil, spleen, soft tissue, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, urine, stool, saliva, cerebrospinal fluid, pericardial fluid, peritoneal fluid, placental extracts, amniotic fluid, a cryoprecipitate, a cryosupernatant, a cell lysate, mammalian cell culture or culture medium, products of fermentation, ascitic fluid, proteins present in blood cells, solid tumours, or any other specimen, or any extract thereof, obtained from a patient (human or animal), test subject, or experimental animal. In some embodiments, it may be desirable to separate cancerous cells from non-cancerous cells, or cells having DNA damage from undamaged cells, in a sample.

A sample may also include, without limitation, products produced in cell culture by normal or transformed cells (e.g., via recombinant DNA or monoclonal antibody technology). A sample may also include, without limitation, any organ, tissue, cell, or cell extract isolated from a non-mammalian subject, such as an insect or a worm. A “sample” may also be a cell or cell line created under experimental conditions, that is not directly isolated from a subject. A sample can also be cell-free, artificially derived or synthesised. A sample may be from a cell or tissue known to be cancerous or have DNA damage, suspected of being cancerous or having DNA damage, or believed not to be cancerous or have DNA damage (e.g., normal or control).

COP1, p53, or ATM nucleic acid molecule or polypeptide expression or activity, or COP1/ATM or COP1/p53 binding, may be assayed using a variety of techniques, including immunohistochemistry (IHC), in situ hybridization (ISH), Northern blotting, polymerase chain reaction (e.g., real time quantitative PCR or RT-PCR), antibody based assays, such as immunoprecipitation, immunofluorescence, Western blotting, etc. For example, methods such as sequencing, single-strand conformational polymorphism (SSCP) analysis, or restriction fragment length polymorphism (RFLP) analysis of PCR products derived from a sample can be used to detect a mutation in a COP1 or p53 gene; immunoprecipitation, RIA, ELISA or western blotting can be used to measure levels of COP1 or ATM or p53 polypeptide or binding; northern blotting can be used to measure COP1 or p53 mRNA levels, or PCR can be used to measure the level of a COP1 or ATM or p53 nucleic acid molecule. Such assays include detection of any or all forms of COP1 or ATM or p53, including precursors, fragments (e.g., created by endoproteolytic degradation), post-translationally modified forms, etc. The methods of the invention encompass assaying for COP1 related biological activities such as auto-ubiquitination, p53 degradation, ubiquitination of p53, inhibition of p53 transactivation, inhibition of p53 induced apoptosis, reduction of p21 mRNA, etc. The methods of the invention encompass assaying for ATM related biological activities, such as kinase activity.

In some embodiments, cells in a subject may be exposed in vivo to an antibody (e.g., a COP1 antibody, and optionally, an ATM or a p53 antibody) which is optionally detectably labeled e.g., radioactive isotope, and binding of the antibody to the cells may be evaluated by e.g., external scanning for radioactivity or analysis of a biopsy. The assays may be conducted using detectably labelled molecules, i.e., any means for marking and identifying the presence of a molecule, e.g., an oligonucleotide probe or primer, a gene or fragment thereof, a peptide, or a cDNA molecule. Methods for detectably-labelling a molecule are well known in the art and include, without limitation, radioactive labelling (e.g., with an isotope such as ³²p or ³⁵S) and nonradioactive labelling such as, enzymatic labelling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labelled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

In some embodiments, cell in a subject may be exposed in vivo to a COP1 compound as described herein, to modulate the response to DNA damage or p53 activity. The COP1 compound may be a nucleic acid molecule that encodes a COP1 polypeptide or fragment, analog, mimetic, or variant thereof.

Pharmaceutical Compositions, Dosages, And Administration

Compounds of the invention can be provided alone or in combination with other compounds (for example, nucleic acid molecules, small molecules, mimetics, peptides, or peptide analogs), in the presence of a liposome, an adjuvant, or any pharmaceutically acceptable carrier, in a form suitable for administration to mammals, for example, humans, mice, etc. For example, compounds of this invention can be used to modulate the response to DNA damage, p53 activity, COP1 activity or ATM activity in a subject.

In some embodiments, therapeutic compounds according to the invention include COP1 molecules or nucleic acid molecules, or small molecules, mimetics, peptides, or peptide analogs thereof, for example, COP1 polypeptides having a phosphorylatable amino acid residue homologous to S387 of a human COP1 polypeptide. Compounds according to the invention may be provided chronically or intermittently. “Chronic” administration refers to administration of the compound(s) continuously for an extended period of time, instead of administering an acute short term dose, so as to maintain the initial therapeutic effect (activity). “Intermittent” administration is treatment that is interspersed with period of no treatment of that particular compound.

Conventional pharmaceutical practice may be employed to provide suitable formulations or compositions to administer the compounds to subjects suffering from or presymptomatic for a cancer. Any appropriate route of administration may be employed, for example, parenteral, intravenous, subcutaneous, intramuscular, intracranial, intraorbital, ophthalmic, intraventricular, intracapsular, intraspinal, intrathecal, intracisternal, intraperitoneal, intranasal, aerosol, topical, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic or preventative compositions for a neoplastic growth, the compounds can be administered to an individual in an amount sufficient to prevent, inhibit, or slow a DNA damage or cancer growth or progression, depending on the cancer and the desired result. Measures of efficacy of the compound for treatment of a tumor growth include an observable and/or measurable reduction in or absence of one or more of the following: reduction in the number of cells with DNA damage; reduction in the number of cancer cells or absence of the cancer cells, reduction in the tumor size; inhibition (i.e., prevent, inhibit, slow, or stop) of cancer cell infiltration into peripheral organs; inhibition (i.e., prevent, inhibit, slow, or stop) of tumor metastasis; inhibition, to some extent, of tumor growth; and/or relief to some extent, one or more of the symptoms associated with the specific cancer, and reduced morbidity and mortality, or an increase in apoptosis or cell death of cancer cells or cells having DNA damage.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as an observable and/or measurable reduction in or absence of one or more of the following: reduction in the number of cells with DNA damage; reduction in the number of cancer cells or absence of the cancer cells, reduction in the tumor size; inhibition (i.e., prevent, inhibit, slow, or stop) of cancer cell infiltration into peripheral organs; inhibition (i.e., prevent, inhibit, slow, or stop) of tumor metastasis; inhibition, to some extent, of tumor growth; and/or relief to some extent, one or more of the symptoms associated with the specific cancer, and reduced morbidity and mortality, or an increase in apoptosis or cell death of cancer cells or cells having DNA damage. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as an observable and/or measurable reduction in or absence of one or more of the following: reduction in the number of cells with DNA damage; reduction in the number of cancer cells or absence of the cancer cells, reduction in the tumor size; inhibition (i.e., prevent, inhibit, slow, or stop) of cancer cell infiltration into peripheral organs; inhibition (i.e., prevent, inhibit, slow, or stop) of tumor metastasis; inhibition, to some extent, of tumor growth; and/or relief to some extent, one or more of the symptoms associated with the specific cancer, and reduced morbidity and mortality, or an increase in apoptosis or cell death of cancer cells or cells having DNA damage. Typically, a prophylactic dose is used in subjects prior to or at an earlier stage of disease, so that a prophylactically effective amount may be less than a therapeutically effective amount. A preferred range for therapeutically or prophylactically effective amounts of a compound may be any value from 0.1 nM-0.1 nM, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

In some embodiments, a pharmaceutical composition may include a nucleic acid molecule encoding a compound according to the invention (e.g., COP1 polypeptides or nucleic acid molecules, or small molecules, mimetics, peptides, or peptide analogs thereof) in combination with a pharmaceutically acceptable carrier. Such pharmaceutical compositions may be administered using any appropriate technique such as a viral vector system. Suitable viral vectors may include adenovirus, vaccinia virus or other pox virus, herpes virus, etc., and any technique for administration of such vectors can be used.

### EXAMPLE 1

### Materials and Methods

Expression Vectors, Recombinant Proteins, and Antibodies

FLAG-COP1, pcDNA3.1+p53, p21-Luc, bax-Luc, pGEX4T1-p53, and pGEX6P1-COP1 have been previously described^{3,21}. GST-S387 peptides contain amino acids 377-400 of a human COP1 polypeptide and GST-S387-A peptide has an alanine mutation at S387. FLAG-ATM and FLAG-ATM-KD were a kind gift from Professor Michael Kastan (St. Jude Children's Research Hospital, TN). All mutant constructs were generated by Quickchange site-directed mutagenesis (Stratagene). All GST recombinant proteins were expressed in E. coli strain BL21(DE3) codon+ (Stratagene) and subsequently purified using the Glutathione Sepharose 4B batch method (GE Healthcare). Anti-p53 (DO-1) (Calbiochem), anti-p53pS15 (Cell Signalling), anti-p21 (Ab-1) (Calbiochem), anti-FLAG (M2) (Sigma), anti-Myc (9E10) (Roche), anti-actin (ICN), anti-tubulin (Ab6161) (Abcam), anti-histone-H1 (SC8030) (Santa Cruz Biotechnology), anti-GST (RPN1236) (GE Healthcare) and anti-HA (Roche) were used according to manufacturer's recommendations. Anti-COP1 has been previously described^{3,22}. Anti-pS387 is a rabbit polyclonal antibody generated by QCB using the peptide Ac-DSRTA(pS)QLDEFC-NH₂ as the antigen and purified by sequential rounds of affinity purification against phosphorylated and non-phosphorylated peptides.

Cells, Transfections, and Reporter Assays

U2-OS, Saos-2, HEK293T, and H1299 cells were purchased from the ATCC and maintained in McCoy's 5A (Invitrogen) with 10% FBS. GM02052, GM03490, GM0637, and GM09607 fibroblasts were obtained from Coriell Cell Repositories and maintained in MEM (ATCC) with 15% FBS. All transfections were carried out using Lipofectamine 2000 (Invitrogen) according to manufacturer's recommendations. Cytoplasmic and nuclear extraction was carried out using NE-PER (Pierce) kit with GM03490 and GM02052 fibroblasts treated with 10 Gy IR or HEK293 and U2-OS cells transfected with 100 ng of various COP1 constructs. To assess COP1's effect on steady-state levels of p53, Saos-2 cells were transfected with increasing amounts (0.5, 1 and 2 μg) of FLAG-COP1 or FLAG-COP1-S387D with 150 ng pcDNA3.1+p53. For reporter assays, Saos-2 cells were transiently transfected with 150 ng pcDNA3.1+p53, 100 ng p21-Luc, and 10 ng of pRL-TK, with or without increasing amounts (0.5, 1, and 2 μg) of pCMV-FLAG-COP1 or pCMV-FLAG-COP1-S387D. Dual Luciferase assays were carried out according to manufacturer's instructions (Promega).

Immunoprecipitation, GST-Pull Down Assays, and Pulse-Chase Analysis

Cells were lysed in radioimmunoprecipitation assay (RIPA) buffer (0.1% SDS, 1% NP-40, 150 mM NaCl, 0.5% deoxycholate, 50 mM Tris pH7.4, 1 mM DTT and protease inhibitor mix), pre-cleared, and immunoprecipitated with target antibody and protein A/G PLUS beads (Santa Cruz Biotechnology). In vitro pull-down assays were carried out with GST or GST-p53 combined with FLAG-COP1 or FLAG-COP1-S387D purified from mammalian cells in PBST (PBS with 0.1% Tween 20 and 1 mM DTT) and incubated at room temperature for 1 hour with gentle rotation. COP1-bound proteins were eluted with FLAG peptide (Sigma) and were subject to SDS-PAGE and immunoblot with anti-FLAG and anti-GST. Cyclohexamide-chase experiments were carried out by incubating cells with 100 μg/ml CHX and harvested at the indicated time points.

In vitro Ubiquitination and Kinase Assays

FLAG-COP1-WT or FLAG-COP1-S387D were purified from HEK293T cells and incubated with 10 μg Ubiquitin (Boston Biochem), 2 μg Biotinylated-Ubiquitin (Boston Biochem), 20 ng of UbcH5b (A.G. Scientific), 20 ng rabbit E1 (Sigma), in a buffer containing 50 mM Tris pH7.5, 2 mM ATP, 5 mM MgCl₂, 20 μM ZnCl₂, 2 mM DTT and 2 μM ubiquitin aldehyde. Where indicated, 100 ng of GST-p53 purified from E. coli was included in the reaction. After incubation for 2 hours at 30° C. with gentle agitation, reactions were boiled in PBST with 1% SDS for 5 minutes and then reduced to 0.1% SDS with PBST for re-immunoprecipitation with anti-FLAG or anti-p53 (FL393)(Santa Cruz Biotechnology). Finally, samples were subject to SDS-PAGE followed by immunoblotting with Streptavidin-HRP (Invitrogen) to detect ubiquitinated species of COP1 or p53. Kinase assays were carried out as previously described²⁰ with endogenous ATM and exogenous ATM. Wortmannin (Calbiochem) and Caffeine (Sigma) were used as indicated.

Colony Formation Assays

H1299 cells were plated into 10-cm Petri dishes and transfected 24 h later with the indicated expression vectors. Transfected cells were selected in Zeocin (600 μg/ml) for 10 days. The medium was then removed and colonies were washed twice with PBS, fixed in ice-cold methanol for 10 min at −20° C., and then incubated with 0.5% crystal violet solution for 10 minutes upon aspiration of methanol. The plates were subsequently washed with distilled water and allowed to air dry. Stained colonies comprising more than 20 cells were scored and counted under inverted microscope. Three independent experiments were carried out and each count was duplicated.

### EXAMPLE 2

### COP1 is Post-Translationally Modified upon Exposure to IR

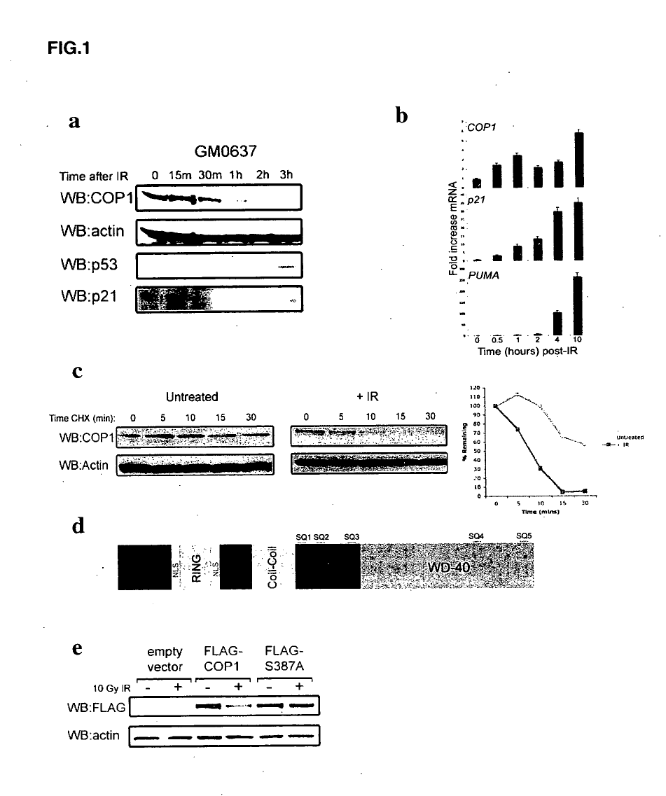

We examined the steady-state levels of COP1 following DNA damage induced by 10 Gy IR using GM0637 fibroblasts. Lysates probed with antibodies to COP1 revealed that as early as 30 minutes post-IR, the steady-state levels of COP1 started to decline and almost vanished by 1 hour (FIG. 1A). Notably the steady-state levels of p53 increased as COP1 levels began to decrease, and this was consistent with the activation of the downstream target gene p21 (FIGS. 1A and 1B). To uncover the mechanism of this reduction in COP1 protein, real-time PCR was implemented to assess gene activity at the COP1 locus. COP1 mRNA levels were modestly increased following IR-stimulation. The p21 and PUMA promoter, 2 classical p53-dependent promoters, were also induced following IR insult albeit at a much larger magnitude than the COP1 promoter. These data therefore imply that the reduction in COP1 protein levels cannot be attributed to a decrease in COP1 mRNA levels. Cyclohexamide (CHX) pulse-chase experiments were therefore conducted to determine if this regulation might be at the post-translational level. Within the GM0637 fibroblasts the COP1 half-life was greater than 30 minutes, whereas this dramatically decreased to just over 7 minutes upon exposure of the cells to 10 Gy IR (FIG. 1C). These data suggest that COP1 is post-translationally modified upon exposure to IR.

### EXAMPLE 3

### COP1 Modification is ATM-Dependent

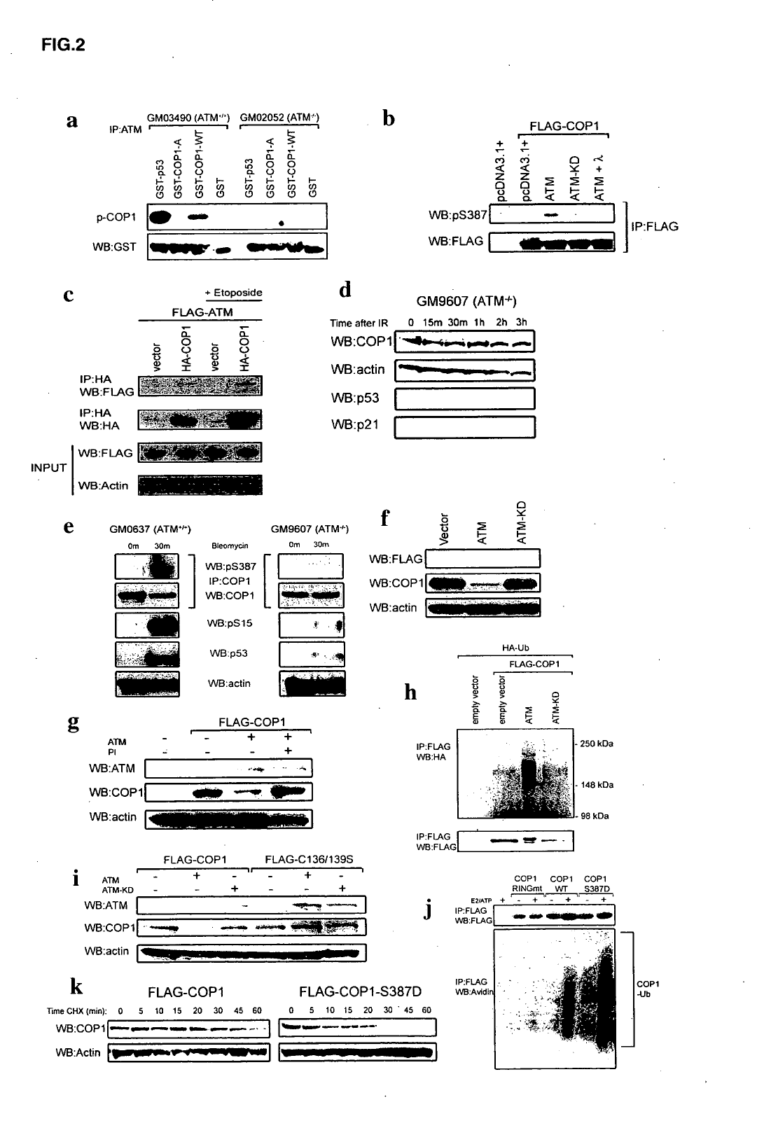

Since ATM is the primary responder to the DNA damage-signalling pathway, potential ATM phosphorylation sites were determined. Five SQ motifs (FIG. 1D) are present on COP1; however, SQ3 (S387) was of primary interest since mutation of this serine residue to alanine prevented the reduction in steady-state levels of COP1 following IR (FIG. 1E). To determine if S387 was a bona fide target for ATM-mediated phosphorylation, GST peptides were purified from E. coli and used as a substrate for an in vitro kinase assay with ATM immunoprecipitated from ATM wild-type or ATM null fibroblasts (FIG. 2A). Indeed, ATM could phosphorylate the peptide harbouring S387 (GST-COP1-WT) and p53 S15 (GST-p53), a classical ATM substrate¹⁷. In contrast, ATM was unable to phosphorylate a peptide containing a S387A mutation (GST-COP1-A). To confirm that full-length COP1 was a substrate for ATM and that S387 was the primary SQ site on COP1, full-length COP1 and COP1-S387A was purified from E. coli and incubated with recombinant ATM or ATM-KD in an in vitro kinase assay (FIG. 5). Incubation of COP1 with ATM, but not ATM-KD, resulted in a robust phospho-signal from COP1 in contrast to the COP1-S387A mutant protein. These data imply that S387 is the major site for ATM in vitro. Therefore, a polyclonal phospho-specific antibody to phosphorylated S387 was raised in rabbits, purified, and characterised by peptide ELISA (FIG. 7) and an in vitro kinase assay with ATM (FIG. 8). To determine if ATM can phosphorylate COP1 in vivo, HEK293T cells were transfected with FLAG-COP1 with, or without, ATM or ATM-KD. Lysates were immunoprecipitated with the FLAG antibody and subjected to western blotting with the anti-pS387 antibody (FIG. 2B). A specific band corresponding to pS387 COP1 was only detected when ATM was co-transfected and disappeared with λ-phosphatase treatment.

To determine if an interaction between ATM and COP1 exists in vivo, HEK293T cells were transfected with FLAG-ATM exclusively, or with HA-COP1 and subjected to etoposide-induced DNA damage or DMSO vehicle control (FIG. 2C). Only when HA-COP1 was transfected with FLAG-ATM, was ATM pulled-down with the anti-HA beads albeit weakly. However, the amount of ATM pulled down with HA-COP1 was strikingly enhanced upon treatment of cells with etoposide, suggesting that the interaction between COP1 and ATM is DNA-damage inducible. To assess if the steady-state level reduction and increased turnover of COP1 post-IR (FIGS. 1A and 1C, respectively) was attributable to the presence of ATM. Fibroblasts derived from A-T patients (GM09607) were treated with IR and harvested at various time points for western blotting. Interestingly, the reduction in steady state levels of COP1 protein was defective in the ATM null fibroblasts (FIG. 2D). This correlated with the absence of p53 stabilisation and induction of p21 protein. The anti-pS387 antibody was then used to probe for ATM-phosphorylated COP1 in ATM-WT and A-T fibroblasts (FIG. 2E). Notably, COP1 pS387 was detected only in the presence of the DNA-damage inducible drug bleomycin within the ATM-WT fibroblasts. p53 pS15 was detected in ATM-WT fibroblasts, whereas it was barely detectable in A-T fibroblasts. pS387 COP1 could not be detected in A-T cells suggesting that modification at this site is ATM-dependent.

### EXAMPLE 4

### ATM Phosphorylation at S387 is Sufficient to Increase the Turnover of COP1 by Promoting Auto-Ubiquitination

Exogenous ATM was introduced into Saos-2 cells and the steady-state levels of exogenous and endogenous COP1 proteins were assessed. Introduction of exogenous ATM caused a dramatic reduction in exogenous and endogenous COP1 protein levels (FIG. 2F and FIG. 9, respectively) in contrast to ATM-KD, which failed to have any effect on steady-state levels of either exogenous or endogenous COP1. Since COP1 is turned over following exposure to IR, we wished to ascertain if this turnover mediated by ATM is via the 26S proteasome. Saos-2 cells were consequently treated with proteasome inhibitor following transfection with HA-COP1 and FLAG-ATM (FIG. 2G). Transfection of ATM caused a striking reduction in COP1; however, this was completely abrogated upon treatment of cells with proteasome inhibitor signifying that ATM phosphorylation of COP1 is promoting proteasome-dependent degradation of COP1. We investigated if COP1 was ubiquitinated in response to activation of ATM. H1299 cells were transfected with HA-tagged ubiquitin, ATM or ATM-KD, and COP1 and were pre-treated with proteasome inhibitor to allow for accumulation of ubiquitinated products before harvesting lysates. COP1 was immunoprecipitated with anti-FLAG and ubiquitinated species were detected with anti-HA (FIG. 2H). Transfection of COP1 alone revealed a low abundance of detectable ubiquitin products; however, when ATM was co-transfected with COP1 there was a substantial signal representing ubiquitinated COP1, in contrast to co-transfection with ATM-KD. In view of the fact that ATM promotes ubiquitination of COP1 and subsequent degradation, we wished to determine whether ATM could signal degradation of a RING mutant of COP1 (C136/139S), with the rationale being that if COP1 were auto-ubiquitinating itself then ATM would be unable to promote COP1 degradation of a RING mutant. Saos-2 cells were transfected with either FLAG-COP1 or FLAG-C136/139S and ATM or ATM-KD, and steady-state level of COP1 proteins were assessed by immunoblotting (FIG. 21). As identified in previous experiments, ATM caused a sharp decrease in COP1 steady state levels, whereas ATM-KD had no effect. In contrast, the RING mutant was refractory to ATM-induced degradation. These data imply that ATM phosphorylation promotes auto-ubiquitination and subsequent degradation of COP1. To further address this question, the COP1-S387D mutant was generated, to mimic phosphorylation on this serine residue, and purified from mammalian cells (FIG. 10) for an in vitro auto-E3 ubiquitination assay using COP1-WT and COP1-RING mutant as positive and negative controls, respectively (FIG. 2J). COP1-WT possessed modest auto-E3 activity, whereas the RING mutant of COP1 was unable to ubiquitinate itself. The S387D COP1 mutant, in contrast, harboured striking auto-E3 ligase activity and is consistent with the notion that ATM phosphorylation increases COP1 ubiquitination via an auto-E3 mechanism. Given that the S387D mutant has increased auto-E3 ligase activity in vitro, it would suggest that this mutant should be turned over at a faster rate in vivo. To address this possibility, CHX pulse-chase experiments were carried out in U2-OS cells transfected with FLAG-COP1 or FLAG-S387D and cells were collected at the indicated time points after incubation with CHX (FIG. 2K). Western blotting of lysates revealed that the half-life of transfected COP1 was ˜50 minutes, whereas the S387D mutant was rapidly turned over, in comparison, with a half-life of ˜15 minutes. Therefore, these data indicate that ATM phosphorylation at S387 is sufficient to increase the turnover of COP1 by promoting auto-ubiquitination.

### EXAMPLE 5

### Modification of COP1 by ATM on S387 is Sufficient to Promote a Cytoplasmic Pool of COP1 in Response to DNA Damage

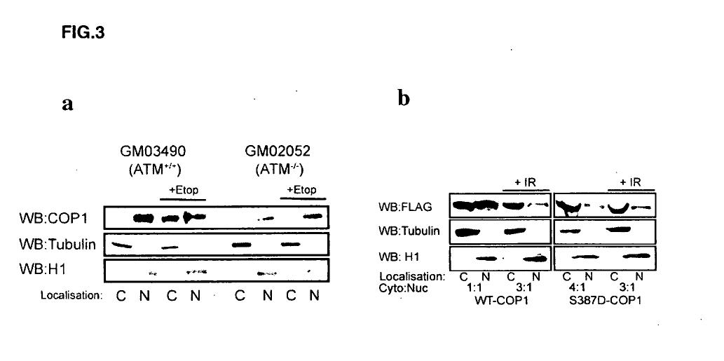

We examined the localisation of exogenous COP1 pre- and post-IR in H1299 cells using immunofluorescence (FIG. 11). Localisation analysis revealed that COP1 was primarily localised to the nuclear compartment as assessed by co-staining with DAPI and overlapping images. Surprisingly, COP1 was primarily localised to the cytoplasmic compartment upon exposure to 10 Gy IR for 2 hours. Next we assessed the localisation of endogenous COP1 by biochemical fractionation within primary fibroblasts either wild-type (GM03490) or truncated for ATM (GM02052). In agreement with the exogenous COP1 studies in H1299 cells (FIG. 10), endogenous COP1 was exclusively localised to the nuclear fraction in wild-type fibroblasts and A-T fibroblasts without any DNA damaging agent (FIG. 3A). Conversely, upon treatment with etoposide approximately 50% of COP1 was present in the cytoplasmic fraction of the GM03490 fibroblasts, whereas no detectable cytoplasmic COP1 was detected from the A-T fibroblasts. These data suggest that the localisation of COP1 within the cytoplasmic compartment is ATM-dependent. With ATM having the ability to phosphorylate COP1 on S387, we wished to determine if this modification at S387 would be sufficient for cytoplasmic localisation. The localisation of COP1-S387D was therefore analysed before and after 10 Gy IR and compared to wild-type COP1 in HEK293T cells (FIG. 3B). WT-COP1 was localised to the cytoplasmic and nuclear compartments at a ratio of approximately 1:1. In contrast, the COP1-S387D protein displayed a striking cytoplasmic/nuclear localisation ratio of 4:1 without any addition of DNA damaging agent. In addition, treatment of cells with IR failed to enhance further the ratio of cytoplasmic/nuclear COP1-S387D, whereas the WT-COP1 cytoplasmic/nuclear ratio increased to approximately 3:1 from 1:1. These data suggest that modification of COP1 by ATM on S387 is sufficient to promote a cytoplasmic pool of COP1 in response to DNA damage.

### EXAMPLE 6

### COP1-S387D is Impaired at Degrading p53 and Repressing p53-Dependent Gene Transactivation

H1299 cells (p53^{-l-}) were transfected with constructs encoding FLAG-COP1 with, or without, HA-p53 and treated with bleomycin for 2 hours and the interaction between COP1 and p53 determined by immunoprecipitation (FIG. 4A). Western blotting for COP1 with the anti-FLAG antibody revealed binding to the HA-beads only when HA-p53 was co-transfected; however, this interaction was significantly inhibited upon DNA damage induced by bleomycin. Similar results were obtained with the reverse IP where HA-p53 was immunoprecipitated with anti-FLAG beads only when FLAG-COP1 was co-transfected (FIG. 4B). Given that COP1 is phosphorylated at S387 following DNA damage, we wished to determine if this modification might play a role in the disruption of the p53/COP1 complex. To this end, we assessed if the COP1-S387D mutant retained the ability to form a complex with p53 relative to WT-COP1 in cells by immunoprecipitation (FIG. 4D). Western blotting of the IPs revealed that significantly less p53 (˜3 fold as assessed by densitometry) was able to complex with COP1-S387D in contrast to WT-COP1. Furthermore, we were able to reconstitute these observations in vitro using purified COP1-S387D (FIG. 9) and E. coli-derived and purified GST-p53 where a ˜5-fold reduction in p53 binding was detected (FIG. 4D). While not completely ablating the interaction between COP1 and p53, it is clear that phosphorylation of S387 renders COP1 defective at binding p53 by at least 3-fold in vitro and in vivo. In addition, this correlated with the decreased ability of COP1-S387D to ubiquitinate p53 in vitro (FIG. 4E). These observations would therefore suggest that modification of COP1 on S387 by ATM might inhibit COP1 's ability to inhibit p53 tumour suppressor function. Experiments were therefore carried out to determine if COP1-S387D retained the ability to degrade p53 in a transient transfection assay. Saos-2 cells were transfected with p53 with an increasing titration of COP1-WT or COP1-S387D (FIG. 4F). Lysates harvested and western blotted with anti-p53 revealed a rapid reduction in p53 steady-state levels upon co-transfection of COP1-WT, whereas there was only a slight reduction in p53 levels upon co-transfection of COP1-S387D. These data also correlated with the levels of p21 protein and p21 gene transactivation ability (FIG. 4F and FIG. 4G, respectively). Therefore, these data imply that COP1-S387D is impaired at degrading p53 and repressing p53-dependent gene transactivation. To verify that this correlates with an appropriate physiological readout, a colony formation assay was performed to monitor the long-term effects of COP1-WT and COP1-S387D at inhibiting p53-dependent function (FIG. 4H) in H1299 cells. Transfection of p53 resulted in few surviving colonies whereas co-transfection of COP1 with p53 resulted in the rescue of a significant number of colonies (25×10³ cfu), relative to p53-only (6×10³ cfu), and COP1-WT-only transfections (72×10³ cfu). In sharp contrast, the COP1-S387D failed to recover a significant number of colonies upon co-transfection with p53 (6×10³ cfu) relative to p53-only (6×10³ cfu) and COP1-S387D-only (77×10³ cfu).

### REFERENCES

The following publications are incorporated by reference herein.
1. Chun, H. H. & Gatti, R. A. Ataxia-telangiectasia, an evolving phenotype. DNA Repair (Amst) 3, 1187-96 (2004).
2. Shiloh, Y. ATM and related protein kinases: safeguarding genome integrity. Nat Rev Cancer 3, 155-68 (2003).
3. Dornan, D. et al. COP1, the negative regulator of p53, is overexpressed in breast and ovarian adenocarcinomas. Cancer Res 64, 7226-30 (2004).
4. Dornan, D. et al. The ubiquitin ligase COP1 is a critical negative regulator of p53. Nature 429, 86-92 (2004).
5. Corcoran, C. A., Huang, Y. & Sheikh, M. S. The p53 paddy wagon: COP1, Pirh2 and MDM2 are found resisting apoptosis and growth arrest. Cancer Biol Ther 3, 721-5 (2004).
6. Bianchi, E. et al. Characterization of human constitutive photomorphogenesis protein 1, a RING finger ubiquitin ligase that interacts with Jun transcription factors and modulates their transcriptional activity. J Biol Chem 278, 19682-90 (2003)
7. Wang H, Kang D, Deng X-W, and Wei, N: Evidence for functional conservation of a mammalian homologue of the light-responsive plant protein COP1. Current Biology 9:711-714, 1999.
8. Ausubel et al., Current Protocols in Molecular Biology, John Wiley & Sons, New York, N.Y., 1998
9. Bartkova, J. et al. DNA damage response as a candidate anti-cancer barrier in early human tumorigenesis. Nature 434, 864-70 (2005).
10. Donehower, L. A. et al. Deficiency of p53 accelerates mammary tumorigenesis in Wnt-1 transgenic mice and promotes chromosomal instability. Genes Dev 9, 882-95 (1995).
11. Liu, G. et al. Chromosome stability, in the absence of apoptosis, is critical for suppression of tumorigenesis in Trp53 mutant mice. Nat Genet 36, 63-8 (2004).
12. Banin, S. et al. Enhanced phosphorylation of p53 by ATM in response to DNA damage. Science 281, 1674-7 (1998).
13. Dumaz, N. & Meek, D. W. Serine 15 phosphorylation stimulates p53 transactivation but does not directly influence interaction with HDM2. Embo J 18, 7002-10 (1999).
14. Khosravi, R. et al. Rapid ATM-dependent phosphorylation of MDM2 precedes p53 accumulation in response to DNA damage. Proc Natl Acad Sci USA 96, 14973-7 (1999).
15. Maya, R. et al. ATM-dependent phosphorylation of Mdm2 on serine 395: role in p53 activation by DNA damage. Genes Dev 15, 1067-77 (2001).
16. Pereg, Y. et al. Phosphorylation of Hdmx mediates its Hdm2- and ATM-dependent degradation in response to DNA damage. Proc Natl Acad Sci USA 102, 5056-61 (2005).
17. Siliciano, J. D. et al. DNA damage induces phosphorylation of the amino terminus of p53. Genes Dev 11, 3471-81 (1997).
18. Myers and Miller, CABIOS, 1989, 4:11-17
19. Yi, C., Wang, H., Wei, N. & Deng, X. W. An initial biochemical and cell biological characterization of the mammalian homologue of a central plant developmental switch, COP1. BMC Cell Biol 3, 30 (2002).
20. Bakkenist, C. J. & Kastan, M. B. DNA damage activates ATM through intermolecular autophosphorylation and dimer dissociation. Nature 421, 499-506 (2003).
21. Dornan, D. et al. Interferon regulatory factor 1 binding to p300 stimulates DNA-dependent acetylation of p53. Mol Cell Biol 24, 10083-98 (2004).
22. Wertz, I. E. et al. Human De-etiolated-1 regulates c-Jun by assembling a CUL4A ubiquitin ligase. Science 303, 1371-4 (2004).
23. Altschul, S. F. Amino acid substitution matrices from an information theoretic perspective. Journal of Molecular Biology, 219: 555-665 (1991).
24. Dayhoff, M. O., Schwartz, R. M., Orcutt, B. C. A model of evolutionary change in proteins In Atlas of Protein Sequence and Structure, 5(3) M. O. Dayhoff (ed.), 345-352, National Biomedical Research Foundation, Washington (1978).
25. States, D. J., Gish, W., Altschul, S. F. Improved Sensitivity of Nucleic Acid Database Search Using Application-Specific Scoring Matrices, Methods: A companion to Methods in Enzymology 3(1): 66-77 (1991).
26. Steven Henikoff and Jorja G. Henikoff. Amino acid substitution matrices from protein blocks.” Proc. Natl. Acad. Sci. USA. 89(Biochemistry): 10915-10919 (1992 ).
27. M. S. Johnson and J. P. Overington. A Structural Basis of Sequence Comparisons: An evaluation of scoring methodologies. Journal of Molecular Biology. 233: 716-738 (1993).
28. Steven Henikoff and Jorja G. Henikoff. Performance Evaluation of Amino Acid Substitution Matrices. Proteins: Structure, Function, and Genetics. 17: 49-61 (1993)
29. Karlin, S. and Altschul, S. F. Methods for assessing the statistical significance of molecular sequence features by using general scoring schemes Proc. Natl. Acad. Sci. USA. 87: 2264-2268 (1990).
30. Eisenberg et al., J. Mol. Bio. 179:125-142, 184).
31. Sambrook, et al., Molecular Cloning: A Laboratory Manual. 2^{nd} ed., Cold Spring Harbor Laboratory, Cold Spring Harbor Laboratory Press, Cold Spring Harbor, N.Y., (1989).

### OTHER EMBODIMENTS

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Accession numbers, as used herein, refer to Accession numbers from multiple databases, including GenBank, the European Molecular Biology Laboratory (EMBL), the DNA Database of Japan (DDBJ), or the Genome Sequence Data Base (GSDB), for nucleotide sequences, and including the Protein Information Resource (PIR), SWISSPROT, Protein Research Foundation (PRF), and Protein Data Bank (PDB) (sequences from solved structures), as well as from translations from annotated coding regions from nucleotide sequences in GenBank, EMBL, DDBJ, or RefSeq, for polypeptide sequences. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. Also, U.S. Provisional Patent Application No. 60/755,412, filed Dec. 31, 2005 is hereby incorporated by reference herein in its entirety. The invention includes all embodiments and variations substantially as hereinbefore described and with reference to the examples and drawings.

What is claimed is:

1. A method of detecting DNA damage in a cell that comprises an ATM polypeptide, the method comprising:
detecting a COP1 polypeptide in the cell, wherein a phosphorylation of said COP1 polypeptide by said ATM polypeptide, or a reduction in the expression level of said COP1 polypeptide, relative to a control is indicative of DNA damage.

2. The method of claim 1 wherein said ATM polypeptide is a human ATM polypeptide.

3. The method of claim 1 wherein said COP1 polypeptide is a human COP1 polypeptide.

4. The method of any one of claims 1 to 3 wherein said phosphorylation is a serine phosphorylation.

5. The method of any one of claims 1 to 4 wherein said COP1 polypeptide is detected using an antibody that specifically binds said COP1 polypeptide.

6. The method of claim 5 wherein said antibody recognizes a peptide comprising an amino acid sequence substantially identical to amino acid residues 377-400 of a human COP1 polypeptide.

7. The method of claim 5 wherein said antibody recognizes a peptide comprising an amino acid sequence homologous to serine 387 of a human COP1 polypeptide.

8. The method of claim 6 or 7 wherein said peptide is phosphorylated on a phosphorylatable amino acid residue that is homologous to serine 387 of a human COP1 polypeptide.

9. The method of any one of claims 1 to 8 further comprising detecting said ATM polypeptide, wherein binding of said COP1 molecule to said ATM molecule is indicative of DNA damage.

10. The method of any one of claims 1 to 9 further comprising detecting one or more of the group consisting of activation of COP1 E3-ligase activity, activation of COP1 auto-ubiquitination, disruption of a p53-COP1 complex, reduction of COP1-dependent p53 ubiquitination, increase in the cytoplasmic-nuclear ratio of COP1, turnover of COP1 polypeptide, and degradation of COP1 polypeptide.

11. The method of any one of claims 1 to 10 further comprising detecting a p53 molecule in said cell, wherein an increase in the expression level of said p53 molecule, or an increase in a p53 activity, relative to a control is indicative of DNA damage.

12. The method of claim 11 wherein said p53 molecule is a human p53 molecule.

13. The method of claim 11 or 12 wherein said p53 molecule is a wild type p53 molecule.

14. The method of any one of claims 11 to 13 wherein said p53 molecule is a p53 polypeptide.

15. The method of claim 14 wherein said p53 polypeptide is detected using an antibody that specifically binds said p53 polypeptide.

16. The method of any one of claims 11 to 15 wherein said p53 activity is selected from one or more of the group consisting of activation of p53-dependent transactivation, activation of p53-induced apoptosis, activation of a p21 molecule, induction of a p21 promoter, increase in p21 mRNA levels, and induction of a PUMA promoter.

17. The method of any one of claims 1 to 16 wherein said DNA damage is caused by radiation or by a chemical compound.

18. The method of claim 17 wherein said radiation is ionizing radiation or ultraviolet radiation.

19. The method of claim 17 wherein said radiation is from radiation therapy.

20. The method of claim 17 wherein said chemical compound is an alkylating agent or a chemotherapeutic agent.

21. The method of any one of claims 1 to 20 wherein said cell has or is at risk for DNA damage.

22. The method of any one of claims 1 to 21 wherein said cell is a cancer cell.

23. The method of claim 22 wherein said cancer cell is selected from one or more of the group consisting of breast cancer, ovarian cancer, colon cancer, lung cancer, and transitional cell cancer.

24. The method of claim 22 or 23 wherein said cancer cell is obtained from a subject undergoing a cancer therapy.

25. The method of claim 24 wherein said cancer therapy is known to cause or is suspected of causing DNA damage.

26. The method of claim 24 or 25 wherein the subject is a human.

27. A method of enhancing a response to DNA damage in a cell, the method comprising exposing the cell to a compound that enhances degradation of a COP1 polypeptide.

28. The method of claim 27 wherein said compound comprises an ATM molecule.

29. The method of claim 28 wherein said ATM molecule is an activated ATM polypeptide.

30. The method of claim 27 wherein said compound enhances the binding of said COP1 polypeptide to an ATM polypeptide or enhances the phosphorylation of said COP1 polypeptide by an ATM polypeptide.

31. A method of enhancing the interaction of a COP1 polypeptide with an ATM polypeptide, the method comprising contacting said COP1 polypeptide and said ATM polypeptide with a compound that enhances the binding of said COP1 polypeptide to said ATM polypeptide.

32. The method of claim 30 or 31 wherein said binding results in one or more of: degradation of said COP1 polypeptide, activation of COP1 E3-ligase activity, activation of COP1 auto-ubiquitination, disruption of a COP1-p53 complex, reduction of COP1-dependent p53 ubiquitination, increase in the cytoplasmic/nuclear ratio of COP1 polypeptides, and increase in the expression levels of p53 molecules.

33. The method of claim 31 wherein the contacting enhances a response to DNA damage in a cell.

34. The method of any one of claims 27 to 30 and 33 wherein the cell has or is at risk for DNA damage.

35. The method of claim 34 wherein said cell is an A-T cell.

36. The method of claim 34 wherein said cell is a cancer cell.

37. The method of any one of claims 27 to 30 and 33 to 36 wherein said response to DNA damage comprises cell apoptosis or p53 activation.

38. The method of claim 37 wherein said p53 activation is selected from one or more of the group consisting of activation of p53-dependent transactivation, activation of p53-induced apoptosis, activation of a p21 molecule, induction of a p21 promoter, and induction of a PUMA promoter.

39. The method of any one of claims 26 to 38 wherein said COP1 polypeptide is a human COP1 polypeptide

40. The method of any one of claims 28 to 39 wherein said ATM polypeptide is a human ATM polypeptide

41. The method of any one of claims 27 to 40 wherein said compound enhances the phosphorylation of an amino acid residue homologous to serine 387 of a human COP1 polypeptide.

42. The method of claim 41 wherein said compound enhances the phosphorylation of serine 387 of human COP1 polypeptide.

43. The method of any one of claims 27 to 42 wherein said compound comprises a COP1 polypeptide or fragment thereof comprising a residue homologous to serine 387 of a human COP1 polypeptide.

44. The method of any one of claims 27 to 42 wherein said compound comprises a polypeptide comprising a sequence substantially identical to amino acid residues 377-400 of a human COP1 polypeptide.

45. The method of claim 43 or 44 wherein said compound comprises a phosphorylation on an amino acid residue that is capable of being phosphorylated.

46. The method of claim 45 wherein said amino acid residue is homologous to serine 387 of a human COP1 polypeptide.

47. The method of any one of claims 27 to 42 wherein said compound comprises a COP1 polypeptide comprising a substitution of threonine, glutamate or aspartate for serine at a residue homologous to serine 387 of a human COP1 polypeptide.

48. The method of any one of claims 27 to 42 wherein said compound comprises a COP1 mimetic compound.

49. A method of identifying a compound that enhances a response to DNA damage in a cell comprising an ATM molecule, the method comprising incubating a COP1 polypeptide in the presence or absence of a test compound under a condition suitable for promoting DNA damage in the cell and determining whether degradation of said COP1 polypeptide is enhanced in the presence of said test compound, wherein a compound that enhances the degradation of said COP1 polypeptide is a compound that enhances a response to DNA damage.

50. The method of claim 49 wherein said determining is done relative to a control.

51. The method of claim 50 wherein said ATM molecule is capable of phosphorylating said COP1 polypeptide.

52. The method of any one of claims 49 to 51 further comprising determining whether one or more of the group consisting of activation of COP1 E3-ligase activity, activation of COP1 auto-ubiquitination, disruption of a COP1-p53 complex, reduction of COP1-dependent p53 ubiquitination, increase in the cytoplasmic/nuclear ratio of COP1 polypeptides, increase in p53 activity, and increase in the expression levels of p53 molecule is enhanced by said compound, wherein such enhancing indicates that said compound is a compound that enhances a response to DNA damage.

53. The method of claim 52 wherein said p53 activity is selected from one or more of the group consisting of activation of p53-dependent transactivation, activation of p53-induced apoptosis, activation of a p21 molecule, induction of a p21 promoter, and induction of a PUMA promoter.

54. The method of any one of claims 49 to 53 wherein the condition suitable for promoting DNA damage is radiation.

55. The method of any one of claims 49 to 54 wherein the response to DNA damage comprises cell apoptosis or p53 activation.

56. A method for identifying a compound that enhances the interaction of a COP1 polypeptide with an ATM polypeptide, the method comprising:
a) incubating a COP1 polypeptide with an ATM polypeptide in the presence or absence of a test compound; and
b) determining whether the test compound increases or stabilizes the binding of said COP1 polypeptide to said ATM polypeptide,
wherein a test compound that increases or stabilizes the binding of said COP1 polypeptide to said ATM polypeptide is a compound that enhances the interaction of a COP1 polypeptide with an ATM polypeptide.

57. An isolated peptide consisting essentially of an amino acid sequence substantially identical to or homologous to amino acid residues 377-400 of human COP1.

58. An isolated peptide consisting essentially of an amino acid sequence as shown in FIG. 6.

59. The peptide of claim 57 or 58 wherein said peptide comprises a substitution of a serine in an SQ motif.

60. The peptide of claim 59 wherein said substitution is an aspartate or glutamate substitution.

61. An isolated or recombinant phosphorylated COP1 peptide that comprises a phosphorylation homologous to S387 of a human COP1 polypeptide.

62. An isolated or recombinant COP1 peptide that comprises an aspartate or glutamate substitution at an amino acid residue homologous to S387 of a human COP1 polypeptide.

63. A COP1 mimetic compound of the peptide of any one of claims 57 to 62.

64. A nucleic acid molecule that encodes the peptide of any one of claims 57 to 62 or the mimetic of claim 63.

65. A vector that comprises the nucleic acid molecule of claim 64 operably linked to a promoter.

66. The vector of claim 65 further comprising an ATM nucleic acid molecule operably linked to a promoter.

67. A host cell that comprises the vector of claim 65 or 66.

68. An reagent that specifically binds an amino acid sequence that is phosphorylated on an amino acid residue homologous to serine 387 of human COP1.

69. The reagent according to claim 68, wherein the amino acid sequence is an S387 phosphorylated human COP1.

70. The reagent according to claim 69, wherein the reagent is an antibody.

71. A nucleic acid molecule that encodes the reagent of claim 68 or antibody of claim 70.

72. A vector with comprising the nucleic acid molecule of claim 71, operably linked to a promoter.

73. A host cell that comprises the vector of claim 72.

74. A kit comprising the reagent of claim 68 or antibody of claim 70 together with instructions for detecting a phosphorylated COP1 molecule in a cell.

75. A pharmaceutical composition comprising the peptide of any one of claims 57 to 62 or the mimetic of claim 63 or a nucleic acid sequence encoding the peptide or mimetic.

76. A method of treating DNA damage or cancer in a subject in need thereof, the method comprising administering to the subject a therapeutically effective amount of a peptide of any one of claims 57 to 62 or the mimetic of claim 63.

77. A method of treating DNA damage or cancer in a subject in need thereof, the method comprising administering to the subject a therapeutically effective amount of a nucleic acid according to claim 64.

78. A method of treating DNA damaged cells or cancer in a subject comprising administering to the subject a compound selected from the group consisting of a compound that enhances the binding of a COP1 polypeptide to an ATM polypeptide and a compound that enhances the phosphorylation of said COP1 polypeptide by an ATM polypeptide.

79. The method according to claim 78, wherein the compound is administered in an amount effective to increase the phosphorylation of a COP1 polypeptide in the DNA damaged cells or cancer.

80. A compound identified according the method of claim 49 or claim 56.

81. Use of a peptide according to any one of claims 57 to 62, the mimetic of claim 63 or the compound of claim 80, for the preparation of a medicament for treating DNA damage or cancer in a subject.

82. A mammalian cell comprising a recombinant nucleic acid molecule encoding a COP1 molecule and a recombinant nucleic acid molecule encoding an ATM molecule.

83. The mammalian cell of claim 82 further comprising a recombinant nucleic acid molecule encoding a p53 molecule.

84. The mammalian cell of claim 82 or 83 wherein said ATM molecule is activated.

85. The mammalian cell of any one of claims 82 to 84 wherein said COP1 molecule is constitutively phosphorylated or constitutively unphosphorylated.

86. The mammalian cell of claim 85, wherein the COP1 molecule is constitutively unphosphorylated at S387 due to a mutation at S387 that prevents COP1 phosphorylation at S387.
